(19) United States
(12) Patent Application Publication
(10) Pub. No.: US 2014/0034232 A1
(43) Pub. Date: Feb. 6, 2014

(54) Disposable Absorbent Articles Having Wide Color Gamut Indicia Printed Thereon

(71) Applicant: The Proctor & Gamble Company, Cincinnati, OH (US)

(72) Inventors: Michael Scott Prodoehl, West Chester, OH (US); Hui Yang, Cincinnati, OH (US); Kevin Benson McNeil, Loveland, OH (US); Alrick Vincent Warner, Loveland, OH (US)

(73) Assignee: The Proctor & Gamble Company, Cincinnati, OH (US)

(21) Appl. No.: 14/051,475

(22) Filed: Oct. 11, 2013

Related U.S. Application Data
(63) Continuation of application No. 13/040,686, filed on Mar. 4, 2011.

(51) Int. Cl. A61F 13/15

(52) U.S. Cl. CPC A61F 13/15577
USPC 156/277

(57) ABSTRACT

Aspects of the present disclosure involve absorbent articles having components having printed indicia, and more particularly, web materials having indicia printed with a color palette that exceeds the prior art color palette. Such web materials can be printed during the manufacture of components of absorbent articles. As discussed herein, examples of such printed web materials can be used in the manufacture of printed diaper components, such as for example, backsheets, topsheets, landing zones, fasteners, fastening tabs, ears, absorbent cores, and acquisition layers. The apparatuses and methods discussed herein are also applicable to other types of absorbent articles, such as feminine hygiene products.

### FIELD OF THE INVENTION

The present disclosure relates to disposable absorbent articles, and more particularly, to disposable absorbent articles including indicia having a unique color gamut applied thereto.

### BACKGROUND OF THE INVENTION

Along an assembly line, diapers, feminine hygiene garments, and various types of other disposable absorbent articles may be assembled by adding components to and otherwise modifying advancing, continuous webs of material. Webs of material and component parts used to manufacture diapers may include: backsheets, topsheets, absorbent cores, front and/or back ears, fastener components, and various types of elastic webs and components such as leg elastics, barrier leg cuff elastics, and waist elastics. In some processes, indicia are printed on individual components and/or continuous webs of material used to assemble the absorbent articles.

Some process printing methodologies and apparatuses for absorbent paper products often utilize four colors as the basis for generating the resulting color palette. Such process printing may provide producers and manufacturers of absorbent articles with the ability to print on individual components and/or continuous webs of material at speeds that are commercially viable. While practical, currently available processes for printing on individual components and/or continuous webs of material are often limited to a four color base for printing, and, as a result, are unable to capture as wide of a color palette as a process/apparatus that takes advantage of a larger number of base colors.

Some absorbent paper products may have images and/or indicia printed thereon, wherein the printed images are produced by a relatively high number of process colors and/or spot colors.

For example, the prior art discloses color gamut boundaries defined by the following system of 2-dimensional equations in CIELab coordinates (2-D gamut) for absorbent article backsheets, respectively:

{a*=−19.12 to −41.53;b*=−52.66 to −31.98}→b*=−0.9228a*−70.30

{a*=−41.53 to −46.41;b*=−31.98 to −25.51}→b*=−1.3258a*−87.04

{a*=−46.41 to −57.57;b*=−25.51 to 7.07}→b*=−2.9194a*−161.00

{a*=−57.57 to −60.55;b*=7.07 to 27.68}→b*=−6.9161a*−391.09

{a*=−60.55 to −59.35;b*=27.68 to 38.68}→b*=9.1667a*+582.72

{a*=−59.35 to −54.97;b*=38.68 to 55.49}→b*=3.8379a*+266.46

{a*=−54.97 to −6.32;b*=55.49 to 87.62}→b*=0.6604a*+91.79

{a*=−6.32 to 44.02;b*=87.62 to 64.58}→b*=−0.4577a*+84.73

{a*=44.02 to 59.83;b*=64.58 to 57.15}→b*=−0.4700a*+85.27

{a*=59.83 to 64.21;b*=57.15 to 52.64}→b*=−1.0297a*+118.76

{a*=64.21 to 65.33;b*=52.64 to 46.49}→b*=−5.4911a*+405.22

{a*=65.33 to 63.85;b*=46.49 to 30.88}→b*=10.5473a*−642.56

{a*=63.85 to 56.98;b*=30.88 to 1.59}→b*=4.2635a*−241.34

{a*=56.98 to 11.84;b*=1.59 to −39.9}→b*=0.9191a*−50.78

{a*=11.84 to −19.12;b*=−39.9 to −52.66}→b*=0.4121a*−44.78

where L* ranges from 0 to 100.

More specifically, prior art backsheets provide the extrapolated color gamut boundaries defined by the following system of 3-dimensional equations in CIELab coordinates (3-D gamut), respectively:

Backsheet Prior Art Gamut

| Vertices Defining Each Face | | | | | | | | | | | | |
|---|---|---|---|---|---|---|---|---|---|---|---|---|
| Vertex 1 | | | Vertex 2 | | | Vertex 3 | | | E a* + F b* + G L* + H = 0 | | | |
| z1 | x1 | y1 | z2 | x2 | y2 | z3 | x3 | y3 | Face Plane Equation Coefficients | | | |
| L* | a* | b* | L* | a* | b* | L* | a* | b* | E | F | G | H |
| 29 | 3.18 | −25.4 | 49.2 | −19.1 | −52.7 | 32.7 | 11.8 | −39.9 | 192.4 | 255.0 | 558.2 | −10335.2 |
| 29 | 3.18 | −25.4 | 49.2 | −19.1 | −52.7 | 56 | −46.4 | −25.5 | −732.2 | −396.8 | −1348.3 | 31388.6 |
| 29 | 3.18 | −25.4 | 32.7 | 11.8 | −39.9 | 24.9 | 0.31 | 0.82 | −35.6 | 25.2 | 185.9 | −4644.4 |
| 29 | 3.18 | −25.4 | 24.9 | 0.31 | 0.82 | 56 | −46.4 | −25.5 | 707.4 | 281.2 | 1302.4 | −32919.5 |
| 49.2 | −19.1 | −52.7 | 32.7 | 11.8 | −39.9 | 59.1 | 57 | 1.59 | 1021.4 | −1562.2 | 708.5 | −97564.9 |
| 49.2 | −19.1 | −52.7 | 59.1 | 57 | 1.59 | 95.8 | −0.14 | 1.81 | 1989.9 | −3359.9 | 3115.5 | −292042.8 |
| 49.2 | −19.1 | −52.7 | 56 | −46.4 | −25.5 | 61.7 | −30.3 | −42.2 | 267.9 | 265.1 | 16.7 | 18261.2 |
| 49.2 | −19.1 | −52.7 | 61.7 | −30.3 | −42.2 | 95.8 | −0.14 | 1.81 | −193.2 | 756.1 | −804.8 | 75686.4 |
| 32.7 | 11.8 | −39.9 | 24.9 | 0.31 | 0.82 | 37.3 | 35.6 | 18 | 635.0 | −130.3 | −1633.7 | 40638.0 |
| 32.7 | 11.8 | −39.9 | 37.3 | 35.6 | 18 | 59.1 | 57 | 1.59 | 1338.2 | −418.6 | −1630.4 | 20702.4 |
| 24.9 | 0.31 | 0.82 | 37.3 | 35.6 | 18 | 56.7 | −59.4 | 38.7 | 80.1 | −1856.6 | 2360.7 | −57355.1 |
| 24.9 | 0.31 | 0.82 | 56.7 | −59.4 | 38.7 | 50.8 | −60.6 | 27.7 | 126.4 | −390.2 | 701.7 | −17212.4 |
| 24.9 | 0.31 | 0.82 | 50.8 | −60.6 | 27.7 | 56 | −46.4 | −25.5 | 1516.2 | 680.3 | 2857.3 | −72261.4 |
| 37.3 | 35.6 | 18 | 52.4 | 64.2 | 52.6 | 59.1 | 57 | 1.59 | 1004.0 | −299.6 | −1212.3 | 14868.1 |
| 37.3 | 35.6 | 18 | 52.4 | 64.2 | 52.6 | 68.8 | −55 | 55.5 | 523.5 | −2277.0 | 4207.7 | −134362.2 |
| 37.3 | 35.6 | 18 | 56.7 | −59.4 | 38.7 | 68.8 | −55 | 55.5 | −77.9 | 1230.8 | −1685.9 | 43408.9 |
| 52.4 | 64.2 | 52.6 | 66.5 | 44 | 64.6 | 62.8 | 57.3 | 25.6 | 503.1 | 113.7 | 627.7 | −71200.6 |
| 52.4 | 64.2 | 52.6 | 66.5 | 44 | 64.6 | 68.8 | −55 | 55.5 | 155.5 | −1340.4 | 1365.5 | −11017.9 |
| 52.4 | 64.2 | 52.6 | 62.8 | 57.3 | 25.6 | 59.1 | 57 | 1.59 | 352.3 | −29.6 | 155.9 | −29231.9 |
| 66.5 | 44 | 64.6 | 62.8 | 57.3 | 25.6 | 95.8 | −0.14 | 1.81 | −1369.3 | −231.0 | −2555.9 | 245034.5 |
| 66.5 | 44 | 64.6 | 95.8 | −0.14 | 1.81 | 91.1 | −5.06 | 87.2 | −2207.4 | −352.3 | −4077.4 | 390866.1 |
| 66.5 | 44 | 64.6 | 91.1 | −5.06 | 87.2 | 68.8 | −55 | 55.5 | 276.8 | −2321.8 | 2680.3 | −40353.4 |
| 62.8 | 57.3 | 25.6 | 59.1 | 57 | 1.59 | 95.8 | −0.14 | 1.81 | −881.6 | 228.8 | −1372.7 | 130936.9 |
| 56.7 | −59.4 | 38.7 | 50.8 | −60.6 | 27.7 | 73.5 | −52.2 | −1.25 | −419.8 | −22.0 | 126.5 | −31239.2 |
| 56.7 | −59.4 | 38.7 | 73.5 | −52.2 | −1.25 | 68.8 | −55 | 55.5 | −763.5 | −12.8 | 294.9 | −61550.0 |
| 50.8 | −60.6 | 27.7 | 73.5 | −52.2 | −1.25 | 56 | −46.4 | −25.5 | 1055.5 | 277.2 | −34.5 | 57990.5 |
| 73.5 | −52.2 | −1.25 | 56 | −46.4 | −25.5 | 61.7 | −30.3 | −42.2 | −429.5 | −315.5 | 295.2 | −44510.8 |
| 73.5 | −52.2 | −1.25 | 61.7 | −30.3 | −42.2 | 95.8 | −0.14 | 1.81 | −877.0 | −1105.2 | 2199.5 | −208787.1 |
| 73.5 | −52.2 | −1.25 | 95.8 | −0.14 | 1.81 | 91.1 | −5.06 | 87.2 | −1917.5 | 135.5 | 4458.7 | −427568.9 |
| 73.5 | −52.2 | −1.25 | 91.1 | −5.06 | 87.2 | 68.8 | −55 | 55.5 | −1411.8 | 172.1 | 2919.3 | −288001.6 |

In some examples, the prior art discloses color gamut boundaries defined by the following system of 2-dimensional equations in CIELab coordinates (2-D gamut) for absorbent article fastening tabs, respectively:

{a*=20.9 to 19.5;b*=17.6 to −7.4}→b*=17.857a*−355.6

{a*=19.5 to 5.6;b*=−7.4 to −23.4}→b*=1.151a*−29.8

{a*=5.6 to −7.4;b*=−23.4 to −20.8}→b*=−0.200a*−22.3

{a*=−7.4 to −17.0;b*=−20.8 to 7.6}→b*=−2.958a*−42.7

{a*=−17.0 to −16.4;b*=7.6 to 17.6}→b*=16.667a*+290.9

{a*=−16.4 to −4.6;b*=17.6 to 29.8}→b*=1.034a*+34.6

{a*=−4.6 to 20.9;b*=29.8 to 17.6}→b*=−0.478a*+27.6

where L* ranges from 0 to 100.

More specifically, prior art fastening tabs provide the extrapolated color gamut boundaries defined by the following system of 3-dimenional equations in CIELab coordinates (3-D gamut), respectively:

Fastening Tabs Prior Art Gamut
Plane equation: Ex + Fy + Gz + H = 0

| Vertices Defining Each Face | | | | | | | | | | | | |
|---|---|---|---|---|---|---|---|---|---|---|---|---|
| Vertex 1 | | | Vertex 2 | | | Vertex 3 | | | E a* + F b* + G L* + H = 0 | | | |
| z1 | x1 | y1 | z2 | x2 | y2 | z3 | x3 | y3 | Face Plane Equation Coefficients | | | |
| L* | a* | b* | L* | a* | b* | L* | a* | b* | E | F | G | H |
| 92.2 | −0.01 | −2.99 | 91.9 | −3.8 | 23.2 | 78.9 | −16.4 | 17.6 | −343.1 | −45.3 | 352.5 | −32649.7 |
| 92.2 | −0.01 | −2.99 | 91.9 | −3.8 | 23.2 | 78.8 | 20.4 | −7.16 | −352.5 | −57.6 | −518.9 | 47684.9 |
| 92.2 | −0.01 | −2.99 | 78.9 | −16.4 | 17.6 | 77 | −7.37 | −20.8 | −551.3 | −152.0 | 444.3 | −41438.6 |
| 92.2 | −0.01 | −2.99 | 77 | −7.37 | −20.8 | 78.8 | 20.4 | −7.16 | 175.2 | −409.6 | 394.1 | −37580.8 |
| 91.9 | −3.8 | 23.2 | 90.5 | −4.63 | 29.8 | 78.9 | −16.4 | 17.6 | −94.0 | 6.7 | 88.3 | −8631.1 |
| 91.9 | −3.8 | 23.2 | 90.5 | −4.63 | 29.8 | 78.8 | 20.4 | −7.16 | −128.5 | −44.4 | −134.9 | 12943.0 |
| 90.5 | −4.63 | 29.8 | 78.9 | −16.4 | 17.6 | 54.2 | −1.9 | 2.59 | 126.9 | −460.4 | 355.0 | −17818.1 |
| 90.5 | −4.63 | 29.8 | 78.8 | 20.4 | −7.16 | 75.1 | 20.9 | 17.6 | 428.0 | 88.0 | 638.3 | −58426.6 |
| 90.5 | −4.63 | 29.8 | 59.1 | 7.86 | 7.14 | 75.1 | 20.9 | 17.6 | −34.3 | −609.2 | 426.7 | −20615.0 |
| 90.5 | −4.63 | 29.8 | 59.1 | 7.86 | 7.14 | 54.2 | −1.9 | 2.59 | −31.4 | 367.7 | −278.2 | 14078.0 |
| 78.9 | −16.4 | 17.6 | 73.2 | −17 | 7.6 | 72.8 | −17.1 | 5.13 | −10.0 | 0.2 | 0.7 | −220.0 |
| 78.9 | −16.4 | 17.6 | 73.2 | −17 | 7.6 | 54.2 | −1.9 | 2.59 | 161.4 | −95.7 | 153.5 | −7769.4 |
| 78.9 | −16.4 | 17.6 | 72.8 | −17.1 | 5.13 | 77 | −7.37 | −20.8 | −208.9 | −56.0 | 136.5 | −13220.5 |
| 73.2 | −17 | 7.6 | 72.8 | −17.1 | 5.13 | 53.9 | −1.45 | 0.84 | 44.9 | −7.5 | 38.8 | −2019.5 |
| 73.2 | −17 | 7.6 | 53.9 | −1.45 | 0.84 | 54.2 | −1.9 | 2.59 | 31.7 | 3.7 | 24.2 | −1261.5 |
| 72.8 | −17.1 | 5.13 | 77 | −7.37 | −20.8 | 64 | 5.63 | −23.4 | 348.8 | 180.6 | 312.2 | −17712.7 |
| 72.8 | −17.1 | 5.13 | 64 | 5.63 | −23.4 | 55.5 | 2.12 | −6.67 | 389.1 | 222.9 | 278.9 | −14818.7 |
| 72.8 | −17.1 | 5.13 | 53.9 | −1.45 | 0.84 | 55.5 | 2.12 | −6.67 | −148.8 | −92.3 | −101.8 | 5353.3 |
| 77 | −7.37 | −20.8 | 71.8 | −1.32 | −22.7 | 64 | 5.63 | −23.4 | 11.8 | 11.0 | 9.6 | −424.2 |
| 77 | −7.37 | −20.8 | 71.8 | −1.32 | −22.7 | 78.8 | 20.4 | −7.16 | 67.8 | −156.2 | 136.3 | −13246.6 |
| 71.8 | −1.32 | −22.7 | 64 | 5.63 | −23.4 | 78.8 | 20.4 | −7.16 | 117.3 | −218.7 | 121.8 | −13561.0 |
| 64 | 5.63 | −23.4 | 78.8 | 20.4 | −7.16 | 59.1 | 7.86 | 7.14 | −531.7 | 104.6 | 414.5 | −21080.7 |
| 64 | 5.63 | −23.4 | 59.1 | 7.86 | 7.14 | 55.5 | 2.12 | −6.67 | −177.5 | 35.8 | 144.2 | −7389.1 |
| 78.8 | 20.4 | −7.16 | 59.1 | 7.86 | 7.14 | 75.1 | 20.9 | 17.6 | 433.1 | −57.5 | −317.5 | 15784.2 |
| 59.1 | 7.86 | 7.14 | 53.9 | −1.45 | 0.84 | 55.5 | 2.12 | −6.67 | −49.1 | −3.9 | 92.5 | −5055.9 |
| 59.1 | 7.86 | 7.14 | 53.9 | −1.45 | 0.84 | 54.2 | −1.9 | 2.59 | 7.2 | 5.3 | −19.1 | 1037.2 |

Without wishing to be limited by theory, it is thought that providing absorbent articles having components printed with a color palette that exceeds the prior art color palette (i.e., a product having more vibrant, intricate, or bright printed pattern thereon) will delight the consumer. Accordingly, it is desired to provide disposable absorbent articles with printed indicia that have a relatively wide color palette.

### SUMMARY OF THE INVENTION

Aspects of the present disclosure involve absorbent articles having components having printed indicia, and more particularly, web materials having indicia printed with a color palette that exceeds the prior art color palette.

In one form, a disposable absorbent article include: a topsheet; a backsheet; and an absorbent core disposed between the topsheet and the backsheet; and an indicia printed directly on at least one of the backsheet, the absorbent core, and the topsheet, the indicia comprising L*a*b* color values, the indicia disposed upon the web substrate being defined by the difference in CIELab coordinate values disposed inside the boundary described by the following system of equations:

{a*=−54.1 to 72.7;b*=131.5 to 145.8}→b*=0.113a*+137.6

{a*=−131.6 to −54.1;b*=89.1 to 131.5}→b*=0.547a*+161.1

{a*=−165.6 to −131.6;b*=28.0 to 89.1}→b*=1.797a*+325.6

{a*=3.6 to −165.6;b*=−82.6 to 28.0}→b*=−0.654a*−80.3

{a*=127.1 to 3.6;b*=−95.1 to −82.6}→b*=−0.101a*−82.3

{a*=72.7 to 127.1;b*=145.8 to −95.1}→b*=−4.428a*+467.7

wherein L* is from 0 to 100; and, the CIELab coordinate values disposed outside the boundary described by the following system of equations:

{a*=−19.12 to −41.53;b*=−52.66 to −31.98}→b*=−0.9228a*−70.30

{a*=−41.53 to −46.41;b*=−31.98 to −25.51}→b*=−1.3258a*−87.04

{a*=−46.41 to −57.57;b*=−25.51 to 7.07}→b*=−2.9194a*−161.00

{a*=−57.57 to −60.55;b*=7.07 to 27.68}→b*=−6.9161a*−391.09

{a*=−60.55 to −59.35;b*=27.68 to 38.68}→b*=9.1667a*+582.72

{a*=−59.35 to −54.97;b*=38.68 to 55.49}→b*=3.8379a*+266.46

{a*=−54.97 to −6.32;b*=55.49 to 87.62}→b*=0.6604a*+91.79

{a*=−6.32 to 44.02;b*=87.62 to 64.58}→b*=−0.4577a*+84.73

{a*=44.02 to 59.83;b*=64.58 to 57.15}→b*=−0.4700a*+85.27

{a*=59.83 to 64.21;b*=57.15 to 52.64}→b*=−1.0297a*+118.76

{a*=64.21 to 65.33;b*=52.64 to 46.49}→b*=−5.4911a*+405.22

{a*=65.33 to 63.85;b*=46.49 to 30.88}→b*=10.5473a*−642.56

{a*=63.85 to 56.98;b*=30.88 to 1.59}→b*=4.2635a*−241.34

{a*=56.98 to 11.84;b*=1.59 to −39.9}→b*=0.9191a*−50.78

{a*=11.84 to −19.12;b*=−39.9 to −52.66}→b*=0.4121a*−44.78

where L* ranges from 0 to 100.

In another form, a disposable absorbent article includes: a topsheet; a backsheet; and an absorbent core disposed between the topsheet and the backsheet; and an indicia printed directly on at least one of the backsheet, the absorbent core, and the topsheet; and wherein the indicia is provided by five or more process colors.

In yet another form, a disposable absorbent article includes: a topsheet; a backsheet; and an absorbent core disposed between the topsheet and the backsheet; and an indicia printed directly on at least one of the backsheet, the absorbent core, and the topsheet, the indicia comprising L*a*b* color values, the indicia disposed upon the web substrate being defined by the CIELab coordinate values disposed inside the boundary described by the MacAdam 3-D gamut and the CIELab coordinate values disposed outside the Backsheet 3-D gamut.

In still another form, a disposable absorbent article includes: a topsheet; a backsheet; and an absorbent core disposed between the topsheet and the backsheet; and an indicia comprising X colors disposed thereon, the indicia being disposed upon at least one of the backsheet, the absorbent core, and the topsheet by a contact printing system adapted to print the X colors upon the web substrate utilizing X-Y printing components where X and Y are whole numbers, 0<Y<X, and X>1, each of the X colors being defined by L*a*b* color values defined by CIELab coordinate values disposed inside the boundary described by the following system of equations:

{a*=−54.1 to 72.7;b*=131.5 to 145.8}→b*=0.113a*+137.6

{a*=−131.6 to −54.1;b*=89.1 to 131.5}→b*=0.547a*+161.1

{a*=−165.6 to −131.6;b*=28.0 to 89.1}→b*=1.797a*+325.6

{a*=3.6 to −165.6;b*=−82.6 to 28.0}→b*=−0.654a*−80.3

{a*=127.1 to 3.6;b*=−95.1 to −82.6}→b*=−0.101a*−82.3

{a*=72.7 to 127.1;b*=145.8 to −95.1}→b*=−4.428a*+467.7

wherein L* is from 0 to 100.

In still another form, a disposable absorbent article includes: a topsheet; a backsheet; and an absorbent core disposed between the topsheet and the backsheet; and an indicia comprising X colors disposed thereon, the indicia being disposed on at least one of the backsheet, the absorbent core, and the topsheet by a contact printing system adapted to print the X colors upon the web substrate utilizing X-Y printing components where X and Y are whole numbers, 0<Y<X, and X>1, each of the X colors being defined by L*a*b* color values defined by CIELab coordinate values disposed inside the boundary described by the MacAdam 3-D gamut.

In still another form, a disposable absorbent article comprising: a topsheet; a backsheet; and an absorbent core disposed between the topsheet and the backsheet; and an indicia printed directly on at least one of the backsheet, the absorbent core, and the topsheet; and wherein the indicia comprises at least one ink disposed thereon and substantially affixed thereto, the at least one ink having a value defined by a 2-D CIELab (L*a*b*) color gamut, the 2-D CIELab (L*a*b*) color gamut being at least about 273% greater than a Backsheet 2-D CIELab (L*a*b*) color gamut.

In still another form, a disposable absorbent article includes: a topsheet; a backsheet; and an absorbent core disposed between the topsheet and the backsheet; and an indicia printed directly on at least one of the backsheet, the absorbent core, and the topsheet; and wherein the indicia comprises at least one ink disposed thereon and substantially affixed thereto, the at least one ink having a value defined by a 3-D CIELab (L*a*b*) color gamut, the 3-D CIELab (L*a*b*) color gamut being at least about 657% greater than a Backsheet 3-D CIELab (L*a*b*) color gamut.

In still another form, a disposable absorbent article includes: a chassis including a topsheet, a backsheet, and an absorbent core disposed between the topsheet and the backsheet; a fastening tab connected with the chassis; and an indicia printed directly on fastening tab comprising L*a*b* color values, the indicia disposed upon the fastening tab being defined by the difference in CIELab coordinate values disposed inside the boundary described by the following system of equations:

{a*=−54.1 to 72.7;b*=131.5 to 145.8}→b*=0.113a*+137.6

{a*=−131.6 to −54.1;b*=89.1 to 131.5}→b*=0.547a*+161.1

{a*=−165.6 to −131.6;b*=28.0 to 89.1}→b*=1.797a*+325.6

{a*=3.6 to −165.6;b*=−82.6 to 28.0}→b*=−0.654a*−80.3

{a*=127.1 to 3.6;b*=−95.1 to −82.6}→b*=−0.101a*−82.3

{a*=72.7 to 127.1;b*=145.8 to −95.1}→b*=−4.428a*+467.7

wherein L* is from 0 to 100; and, the CIELab coordinate values disposed outside the boundary described by the following system of equations:

{a*=20.9 to 19.5;b*=17.6 to −7.4}→b*=17.857a*−355.6

{a*=19.5 to 5.6;b*=−7.4 to −23.4}→b*=1.151a*−29.8

{a*=5.6 to −7.4;b*=−23.4 to −20.8}→b*=−0.200a*−22.3

{a*=−7.4 to −17.0;b*=−20.8 to 7.6}→b*=−2.958a*−42.7

{a*=−17.0 to −16.4;b*=7.6 to 17.6}→b*=16.667a*+290.9

{a*=−16.4 to −4.6;b*=17.6 to 29.8}→b*=1.034a*+34.6

{a*=−4.6 to 20.9;b*=29.8 to 17.6}→b*=−0.478a*+27.6

where L* ranges from 0 to 100.

In still another form, a disposable absorbent article includes: a chassis including a topsheet, a backsheet, and an absorbent core disposed between the topsheet and the backsheet; a fastening tab connected with the chassis; and an indicia comprising X colors disposed thereon, the indicia being disposed upon the fastening tab by a contact printing system adapted to print the X colors upon the web substrate utilizing X-Y printing components where X and Y are whole numbers, 0<Y<X, and X>1, each of the X colors being defined by L*a*b* color values defined by CIELab coordinate values disposed inside the boundary described by the following system of equations:

{a*=−54.1 to 72.7;b*=131.5 to 145.8}→b*=0.113a*+137.6

{a*=−131.6 to −54.1;b*=89.1 to 131.5}→b*=0.547a*+161.1

{a*=−165.6 to −131.6;b*=28.0 to 89.1}→b*=1.797a*+325.6

{a*=3.6 to −165.6;b*=−82.6 to 28.0}→b*=−0.654a*−80.3

{a*=127.1 to 3.6;b*=−95.1 to −82.6}→b*=−0.101a*−82.3

{a*=72.7 to 127.1;b*=145.8 to −95.1}→b*=−4.428a*+467.7

wherein L* is from 0 to 100.

### BRIEF DESCRIPTION OF THE DRAWINGS

FIG. 1 is a graphical representation of exemplary extrapolated MacAdam and Backsheet 2-D color gamuts in CIELab (L*a*b*) coordinates showing the a*b* plane where L*=0 to 100.

FIG. 1a is a graphical representation of exemplary extrapolated MacAdam and Fastening Tabs 2-D color gamuts in CIELab (L*a*b*) coordinates showing the a*b* plane where L*=0 to 100.

FIG. 2 is a graphical representation of exemplary extrapolated Backsheet 3-D color gamut in CIELab (L*a*b*) coordinates.

FIG. 2a is a graphical representation of exemplary extrapolated Fastening Tabs 3-D color gamut in CIELab (L*a*b*) coordinates.

FIG. 3 is an alternative graphical representation of exemplary extrapolated Backsheet 3-D color gamut in CIELab (L*a*b*) coordinates.

FIG. 3a is an alternative graphical representation of exemplary extrapolated Fastening Tabs 3-D color gamut in CIELab (L*a*b*) coordinates.

FIG. 4 is a graphical representation of exemplary extrapolated MacAdam 3-D color gamut in CIELab (L*a*b*) coordinates.

FIG. 5 is an alternative graphical representation of exemplary extrapolated MacAdam 3-D color gamut in CIELab (L*a*b*) coordinates.

FIG. 6 is a perspective view of an exemplary gravure cylinder suitable for producing the product of the present disclosure.

FIG. 7 is a disposable absorbent article in the form of a diaper.

FIG. 8 is a plan view of a diaper including a chassis shown in a flat, unfolded condition, with the portion of the diaper that faces away from a wearer oriented towards the viewer.

### DETAILED DESCRIPTION OF THE INVENTION

“Absorbent article” is used herein to refer to consumer products whose primary function is to absorb and retain soils and wastes. Non-limiting examples of incontinent absorbent articles include diapers, taped diapers, training and pull-on pants, adult incontinence briefs and undergarments, feminine hygiene garments such as panty liners, absorbent inserts, and the like.

“Diaper” is used herein to refer to an absorbent article generally worn by infants and incontinent persons about the lower torso.

The term “pant” (also referred to as “training pant”, “pre-closed diaper”, “diaper-pant”, “pant diaper”, and “pull-on diaper”) refers herein to disposable absorbent articles having a continuous perimeter waist opening and continuous perimeter leg openings designed for infant or adult wearers.

The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

The term “disposed” is used herein to mean that an element(s) is formed (joined and positioned) in a particular place or position as a macro-unitary structure with other elements or as a separate element joined to another element.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “body facing surface” refers to surfaces of absorbent articles and/or components thereof which face a wearer's body when the absorbent articles are worn, and the term “garment facing surface” refers to surfaces of absorbent articles and/or components thereof that face away from a wearer's body when the absorbent articles are worn. Absorbent articles and components thereof, including the topsheet, backsheet, absorbent core, fastening tabs, and any individual materials of their components, have a body facing surface and a garment facing surface.

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a layer or layers or fibrous materials, films and foils such as plastic films or metallic foils that may be used alone or laminated to one or more web, layer, film and/or foil. As such, a web is a substrate.

The term “nonwoven” refers herein to a material made from continuous (long) filaments (fibers) and/or discontinuous (short) filaments (fibers) by processes such as spunbonding, meltblowing, and the like. Nonwovens do not have a woven or knitted filament pattern.

The term “machine direction” (MD) is used herein to refer to the direction of material flow through a process.

The term “cross direction” (CD) is used herein to refer to a direction that is generally perpendicular to the machine direction.

“Base Color,” as used herein, refers to a color that is used in the halftoning printing process as the foundation for creating additional colors. In some non-limiting embodiments, a base color is provided by a colored ink and/or dye. Non-limiting examples of base colors may selected from the group consisting of: cyan, magenta, yellow, black, red, green, and blue-violet.

“Basis Weight”, as used herein, is the weight per unit area of a sample reported in lbs/3000 ft² or g/m².

“Black”, as used herein, refers to a color and/or base color which absorbs wavelengths in the entire spectral region of from about 380 nm to about 740 nm.

“Blue” or “Blue-violet”, as used herein, refers to a color and/or base color which have a local maximum reflectance in the spectral region of from about 390 nm to about 490 nm.

“Cyan”, as used herein, refers to a color and/or base color which have a local maximum reflectance in the spectral region of from about 390 nm to about 570 nm. In some embodiments, the local maximum reflectance is between the local maximum reflectance of the blue or blue-violet and green local maxima.

“Dot gain” is a phenomenon in printing which causes printed material to look darker than intended. It is caused by halftone dots growing in area between the original image (“input halftone”) and the image finally printed upon the web material (“output halftone”).

A “dye” is a liquid containing coloring matter, for imparting a particular hue to web materials, such as a film, nonwoven, cloth, paper, etc. For purposes of clarity, the terms “fluid” and/or “ink” and/or “dye” may be used interchangeably herein and should not be construed as limiting any disclosure herein to solely a “fluid” and/or “ink” and/or “dye.”

A “fluid” is a substance, as a liquid or gas, that is capable of flowing and that changes its shape at a steady rate when acted upon by a force tending to change its shape. Exemplary fluids suitable for use with the present disclosure includes inks, dyes, softening agents, cleaning agents, dermatological solutions, wetness indicators, adhesives, combinations thereof, and the like.

“Green”, as used herein, refers to a color and/or base color which have a local maximum reflectance in the spectral region of from about 491 nm to about 570 nm.

“Halftone” or “halftoning” as used herein, sometimes known to those of skill in the printing arts as “screening,” is a printing technique that allows for less-than-full saturation of the primary colors. In halftoning, relatively small dots of each primary color are printed in a pattern small enough such that the average human observer perceives a single color. For example, magenta printed with a 20% halftone will appear to the average observer as the color pink. The reason for this is because, without wishing to be limited by theory, the average observer may perceive the tiny magenta dots and white paper between the dots as lighter, and less saturated, than the color of pure magenta ink.

“Hue” is the relative red, yellow, green, and blue-violet in a particular color. A ray can be created from the origin to any color within the two-dimensional a*b* space. Hue is the angle measured from 0° (the positive a* axis) to the created ray. Hue can be any value of between 0° to 360°. Lightness is determined from the L* value with higher values being more white and lower values being more black.

An “ink” is a fluid or viscous substance used for writing or printing.

“Lab Color” or “L*a*b* Color Space,” as used herein, refers to a color model that is used by those of skill in the art to characterize and quantitatively describe perceived colors with a relatively high level of precision. More specifically, CIELab may be used to illustrate a gamut of color because L*a*b* color space has a relatively high degree of perceptual uniformity between colors. As a result, L*a*b* color space may be used to describe the gamut of colors that an ordinary observer may actually perceive visually.

A color's identification is determined according to the Commission Internationale de l'Eclairage L*a*b* Color Space (hereinafter “CIELab”). CIELab is a mathematical color scale based on the Commission Internationale de l'Eclairage (hereinafter “CIE”) 1976 standard. CIELab allows a color to be plotted in a three-dimensional space analogous to the Cartesian xyz space. Any color may be plotted in CIELab according to the three values (L*, a*, b*). For example, there is an origin with two axis a* and b* that are coplanar and perpendicular, as well as an L-axis which is perpendicular to the a* and b* axes, and intersects those axes only at the origin. A negative a* value represents green and a positive a* value represents red. CIELab has the colors blue-violet to yellow on what is traditionally the y-axis in Cartesian xyz space. CIELab identifies this axis as the b*-axis. Negative b* values represent blue-violet and positive b* values represent yellow. CIELab has lightness on what is traditionally the z-axis in Cartesian xyz space. CIELab identifies this axis as the L-axis. The L*-axis ranges in value from 100, which is white, to 0, which is black. An L* value of 50 represents a mid-tone gray (provided that a* and b* are 0). Any color may be plotted in CIELab according to the three values (L*, a*, b*). As described herein, equal distances in CIELab space correspond to approximately uniform changes in perceived color. As a result, one of skill in the art is able to approximate perceptual differences between any two colors by treating each color as a different point in a three dimensional, Euclidian, coordinate system, and calculating the Euclidian distance between the two points (ΔE*_{ab}).

The three dimensional CIELab allows the three color components of chroma, hue, and lightness to be calculated. Within the two-dimensional space formed from the a-axis and b-axis, the components of hue and chroma can be determined Chroma, (C*), is the relative saturation of the perceived color and can be determined by the distance from the origin in the a*b* plane. Chroma, for a particular a*, b* set can be calculated as follows:

C*=(a*²+b*²)^{1/2}

For example, a color with a*b* values of (10,0) would exhibit a lesser chroma than a color with a*b* values of (20,0). The latter color would be perceived qualitatively as being “more red” than the former. Hue is the relative red, yellow, green, and blue-violet in a particular color. A ray can be created from the origin to any color within the two-dimensional a*b* space.

“Magenta”, as used herein, refers to a color and/or base color which have a local maximum reflectance in the spectral region of from about 390 nm to about 490 nm and 621 nm to about 740 nm.

“Process Printing,” as used herein, refers to the method of providing color prints using three primary colors cyan, magenta, yellow and black. Each layer of color is added over a base substrate. In some embodiments, the base substrate is white or off-white in color. With the addition of each layer of color, certain amounts of light are absorbed (those of skill in the printing arts will understand that the inks actually “subtract” from the brightness of the white background), resulting in various colors. CMY (cyan, magenta, yellow) are used in combination to provide additional colors. Non-limiting examples of such colors are red, green, and blue. K (black) is used to provide alternate shades and pigments. One of skill in the art will appreciate that CMY may alternatively be used in combination to provide a black-type color.

“Red”, as used herein, refers to a color and/or base color which has a local maximum reflectance in the spectral region of from about 621 nm to about 740 nm.

“Resultant Color,” as used herein, refers to the color that an ordinary observer perceives on the finished product of a halftone printing process. As exemplified herein, the resultant color of magenta printed at a 20% halftone is pink.

“Web materials” include products suitable for the manufacture of articles upon which indicia may be imprinted thereon and substantially affixed thereto. Web materials suitable for use and within the intended disclosure include fibrous structures, absorbent paper products, and/or products containing fibers. Other materials are also intended to be within the scope of the present invention as long as they do not interfere or counter act any advantage presented by the instant invention. Suitable web materials may include foils, polymer sheets, cloth, wovens or nonwovens, paper, cellulose fiber sheets, co-extrusions, laminates, high internal phase emulsion foam materials, and combinations thereof. The properties of a selected deformable material can include, though are not restricted to, combinations or degrees of being: porous, non-porous, microporous, gas or liquid permeable, non-permeable, hydrophilic, hydrophobic, hydroscopic, oleophilic, oleophobic, high critical surface tension, low critical surface tension, surface pre-textured, elastically yieldable, plastically yieldable, electrically conductive, and electrically non-conductive. Such materials can be homogeneous or composition combinations. Also included within the scope of the definition are products suitable for use as packaging materials. This may include, but not be limited to, polyethylene films, polypropylene films, liner board, paperboard, cartoning materials, and the like.

“Yellow”, as used herein, refers to a color and/or base color which have a local maximum reflectance in the spectral region of from about 571 nm to about 620 nm.

“Z-direction” as used herein, is the direction perpendicular to both the machine and cross machine directions.

All percentages and ratios are calculated by weight unless otherwise indicated. Furthermore, all percentages and ratios are calculated based on the total composition unless otherwise stated. Additionally, unless otherwise noted, all component or composition levels are in reference to the active level of that component or composition and are exclusive of impurities; for example, residual solvents or by-products which may be present in commercially available sources.

Aspects of the present disclosure involve absorbent articles having components having printed indicia, and more particularly, web materials having indicia printed with a color palette that exceeds the prior art color palette. Such web materials can be printed during the manufacture of components of absorbent articles. As discussed herein, examples of such printed web materials can be used in the manufacture of printed diaper components, such as for example, backsheets, topsheets, landing zones, fasteners, ears, absorbent cores, and acquisition layers. Additional descriptions of the aforementioned diaper components can be found in U.S. Pat. Nos. 5,569,234; 5,702,551; 5,643,588; 5,674,216; 5,897,545; and 6,120,489; and U.S. Patent Publication Nos. 2010/0300309 and 2010/0089264. Although the description below is mainly related to diaper components, it is to be appreciated that the apparatuses and methods discussed herein are also applicable to other types of absorbent articles, such as feminine hygiene products.

In some embodiments, central impression printing may be used to provide ink to web materials in the form of substrates used in the manufacture of printed absorbent article components. Exemplary central impression printing methods and apparatus are described in U.S. Pat. Nos. 6,220,156; 6,283,024; and 5,083,511. In other embodiments, in-line printing may be used to provide ink to the substrates. Exemplary in-line printing methods and apparatus are described in U.S. Pat. Nos. 6,587,133; 6,026,748; and 5,331,890. Printing may also be performed using any multi-stage printing apparatus for printing on absorbent paper product substrates such as those exemplified in U.S. Pat. Nos. 5,638,752, 6,026,748, and 5,331,890.

In some embodiments, a multi-stage printing system may be utilized. In one embodiment, seven colors can be used to provide the printed substrates of the present disclosure. Surprisingly, it is found that when red, green, and blue-violet inks in particular are used in conjunction with the standard CMYK process colors for a seven-color process printing procedure, the resultant paper products made with this process/apparatus exhibited a noticeably improved appearance and larger color gamut as compared to the prior art four color printing. Without wishing to be limited by theory, it is thought that the additional ink colors provide a larger resultant color palette than is possible from the prior art printing processes/apparatus.

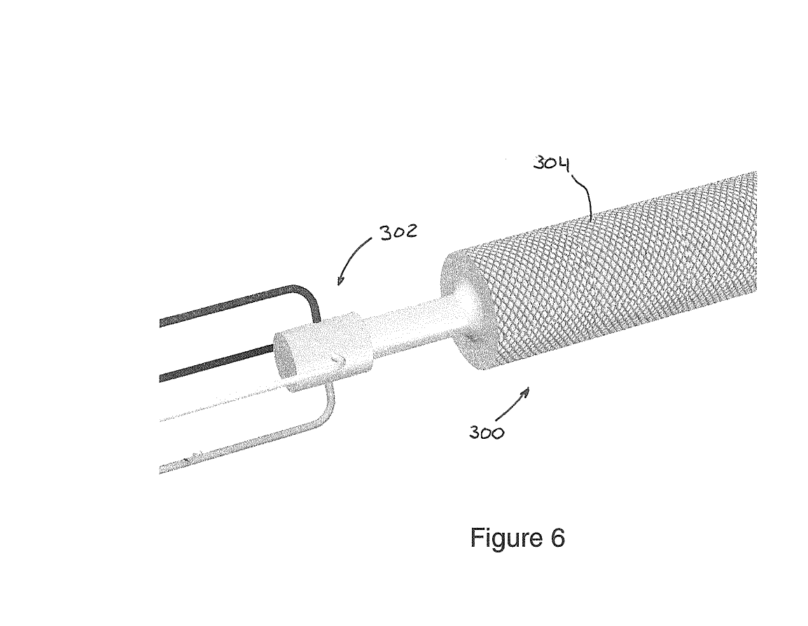

Alternatively, FIG. 6 shows a perspective view of an exemplary, non-limiting, contact printing system 300. Such contact printing systems 300 can be generally formed from printing components that displace a fluid 302 onto a web substrate or article (also known as a central roll or gravure cylinder 304) and any other ancillary components necessary to assist the displacement of the fluid 302 from the central roll 304 onto the web substrate or article in order to, for example, print an image onto the web substrate or article. As shown, an exemplary printing component commensurate in scope with the apparatus of the present disclosure can be a gravure cylinder 304. The exemplary gravure cylinder 304 is used to carry a desired pattern and quantity of fluid 302 (e.g., ink) and transfer a portion of the fluid 302 to a web material or article that has been placed in contact with the gravure cylinder 304 which in turn transfers the fluid 302 to the web material or article. Alternatively, as would be understood by one of skill in the art, the principles of the present disclosure would also apply to a printing plate which in turn can transfer a fluid 302 to a web material. In any regard, printing methods of the present disclosure may ultimately be used to apply a broad range of fluids 302 to a web substrate at a target rate and in a desired pattern. By way of non-limiting example, the contact printing system 300 of the present disclosure incorporating the unique and exemplary gravure cylinder 304 described herein can apply more than just a single fluid 302 (e.g., can apply a plurality of individual inks each having a different color) to a web substrate when compared to a conventional gravure printing system as described herein (e.g., a single central impression cylinder can only apply a single ink). For example, various inks can be mixed in situ to form a virtually unlimited number of colors representing a heretofore unrealizable gamut.

Represented mathematically, the contact printing system 300 described herein can print X colors upon a web substrate utilizing X-Y printing components where X and Y are whole numbers, 0<Y<X, and X>1. In some embodiments, each fluid 302 disposed upon a web substrate in contact with the gravure cylinder 304 is first disposed within the inner portion of the gravure cylinder 304 and directed to those portions of the outer surface 206 of gravure cylinder 304 to form the desired pattern of any indicia to be formed upon a web substrate in contact with gravure cylinder 304. Each fluid 302 may be applied directly to a web substrate or can be combined with another fluid (which may or may not be the resulting combination of other different fluids 302) and applied to a web substrate. Such an exemplary contact printing system is described in co-pending U.S. patent application Ser. No. 13/040,287, entitled “An Apparatus for Applying Indicia having a Large Color Gamut on Web Substrates,” filed on Mar. 4, 2011, which is incorporated by reference herein. In some embodiments, the contact printing system 300 can print at least 2 colors with 1 printing component or at least 3 colors with 1 printing component or at least 4 colors with 1 printing component or at least 5 colors with 1 printing component or at least 6 colors with 1 printing component or at least 7 colors with 1 printing component or at least 8 colors with 1 printing component. In other embodiments, the contact printing system 300 can print at least 3 colors with 2 printing components or at least 4 colors with 2 printing components or at least 8 colors with 2 printing components or at least 4 colors with 3 printing components or at least 16 colors with 2 printing components or at least 16 colors with 3 printing components or at least 24 colors with 3 printing components.

As described herein, one embodiment of the present disclosure is printed using a greater number of base colors than in any prior art printing processes. In one embodiment, the base colors that can be used are: cyan, magenta, yellow, black, red, green, and blue-violet.

In other embodiments, to improve ink rub-off resistance, the ink composition of this invention may contain a wax. A wax suitable for this invention includes but is not limited to a polyethylene wax emulsion. Addition of a wax to the ink composition of the present invention enhances rub resistance by setting up a barrier which inhibits the physical disruption of the ink film after application of the ink to the fibrous sheet. Based on weight percent solids of the total ink composition, suitable addition ranges for the wax are from about 0.5% solids to 10% solids. An example of a suitable polyethylene wax emulsion is JONWAX 26 supplied by S.C. Johnson & Sons, Inc. of Racine, Wis. Glycerin may also be added to the ink composition used in the present invention in order to improve rub-off resistance. Based upon weight percent of the total ink composition, suitable addition ranges for glycerin can range from about 0.5% to 20%, or from about 3% to 15%, or from about 8% to 13%.

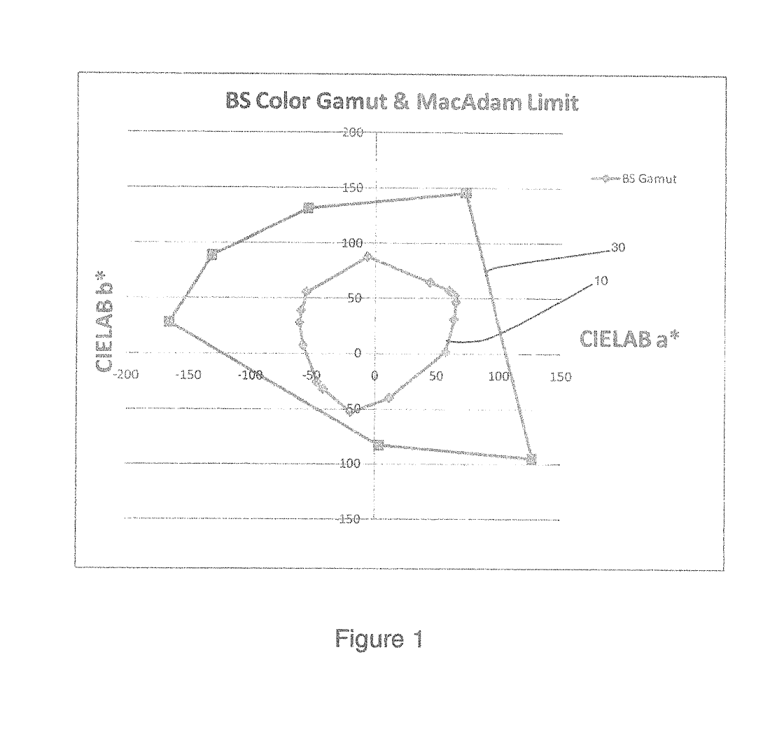

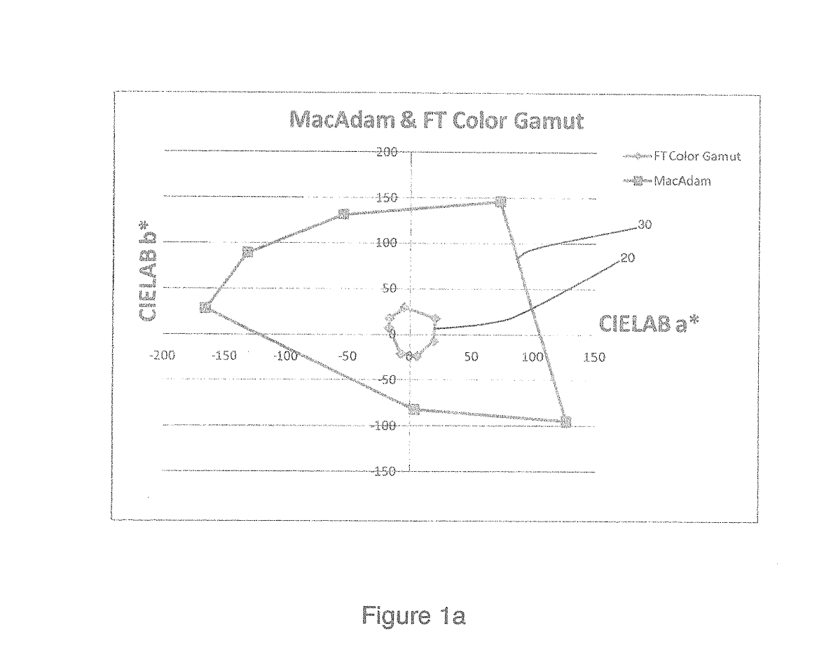

FIG. 1 shows an exemplary extrapolated graphical representation of the 2-dimensional (2-D) color gamut available to prior art backsheet absorbent article substrates in an L*a*b color space in the a*b* plane. The L*a*b* points are chosen according to the Color Test Method described below. Without wishing to be limited by theory, it is thought that the most “intense” (i.e., 100% halftone) colors represent the outer boundaries of the color gamut. Surprisingly, it was found that the prior art Backsheet (“BS”) 2-D color gamut 10 does not occupy as large of an area as the MacAdam 2-D color gamut 30 (the maximum 2-D theoretical human color perception) as applied to absorbent article backsheet substrates of the present disclosure. Stated differently, the combination of the colors available with the MacAdam color gamut 30 provide resultant colors that extend beyond the limitations of the red, green, and blue-violet process colors and well beyond the Backsheet 2-D color gamut 10 colors and color combinations when described in L*a*b* space.

For the 2-D color gamuts discussed herein, the formula (new gamut area—prior art gamut area)/prior art gamut area*100% is used to calculate the percent increase of the area circumscribed by the 2-D gamut plots of the MacAdam color gamut 30 compared to the prior art Backsheet color gamut 10. The area circumscribed by the Backsheet color gamut 10 and the MacAdam color gamut 30 can be determined to be 12,085 and 45,100 relative area units, respectively. Using these values in the equation results in a color gamut percentage increase of about 273% (MacAdam), that is available over the palette of the prior art absorbent paper products—clearly, a surprising result.

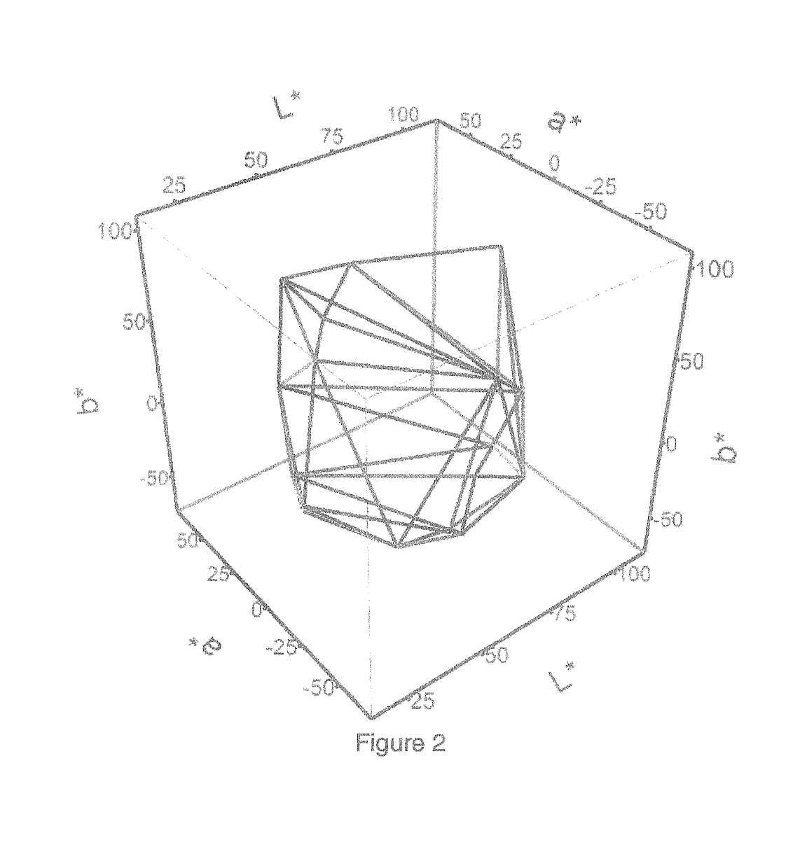

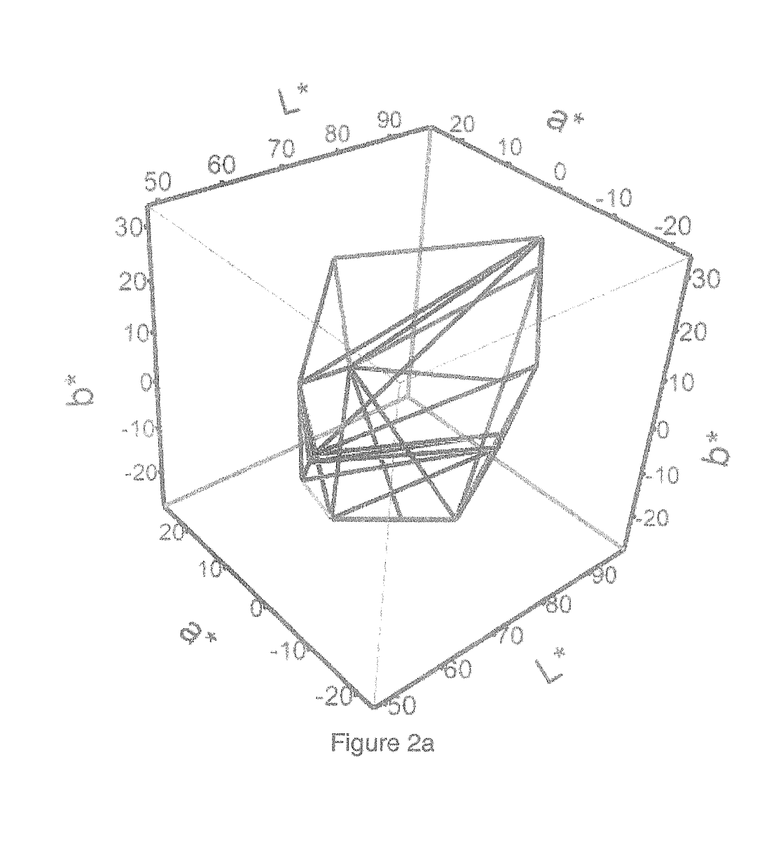

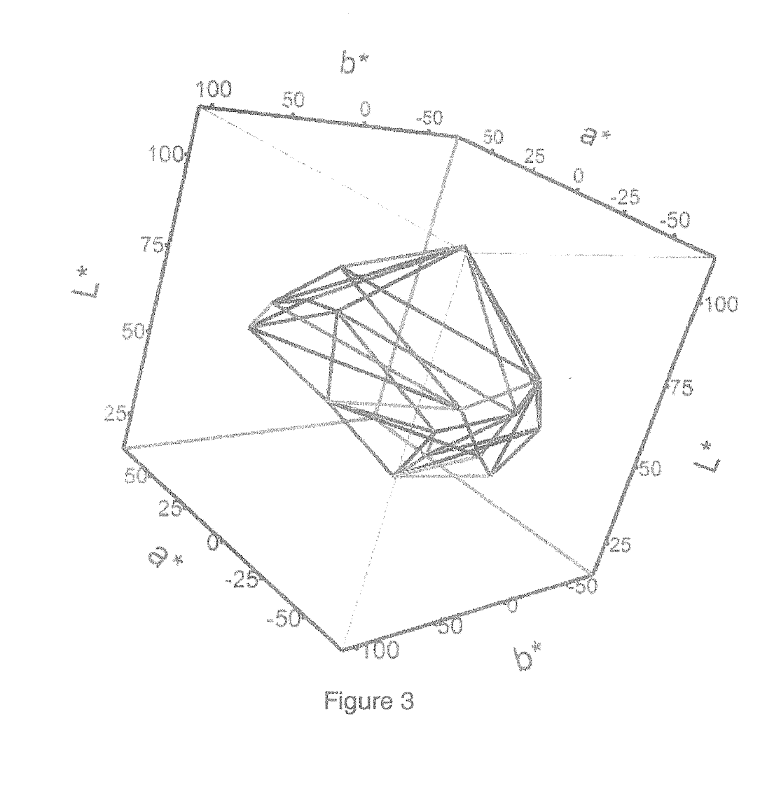

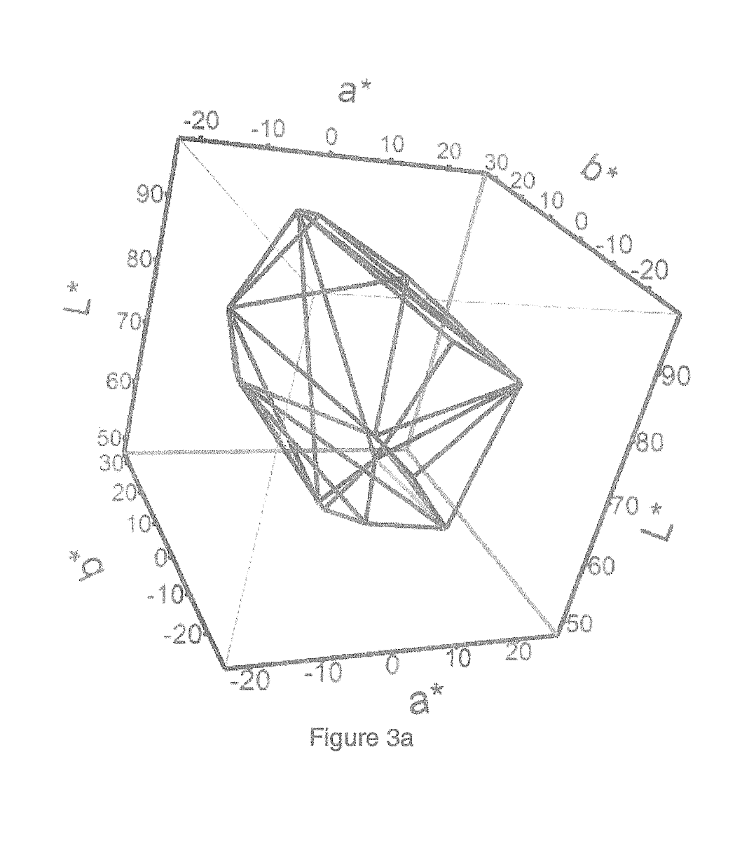

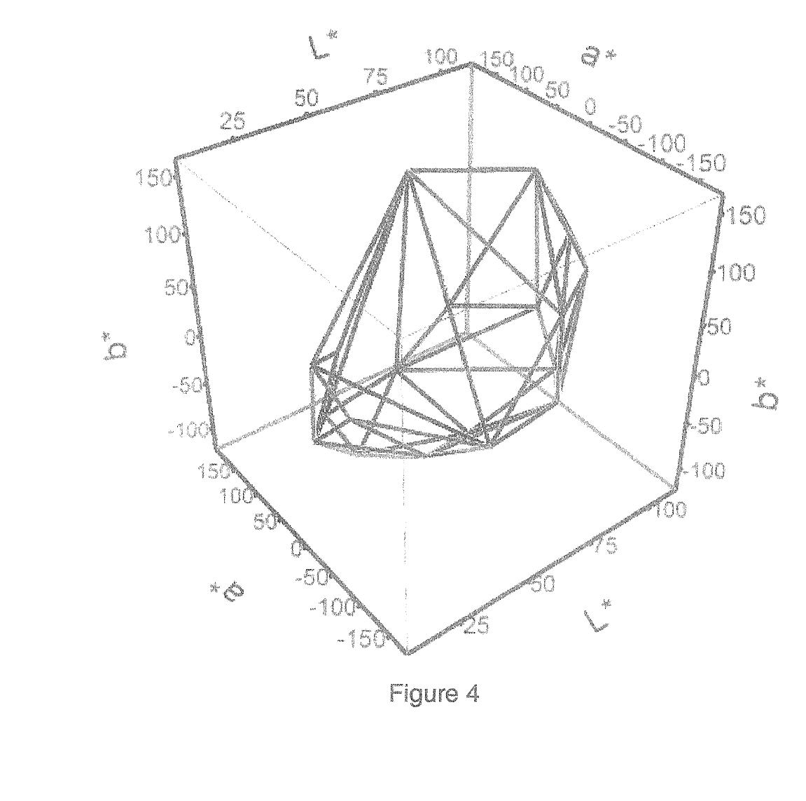

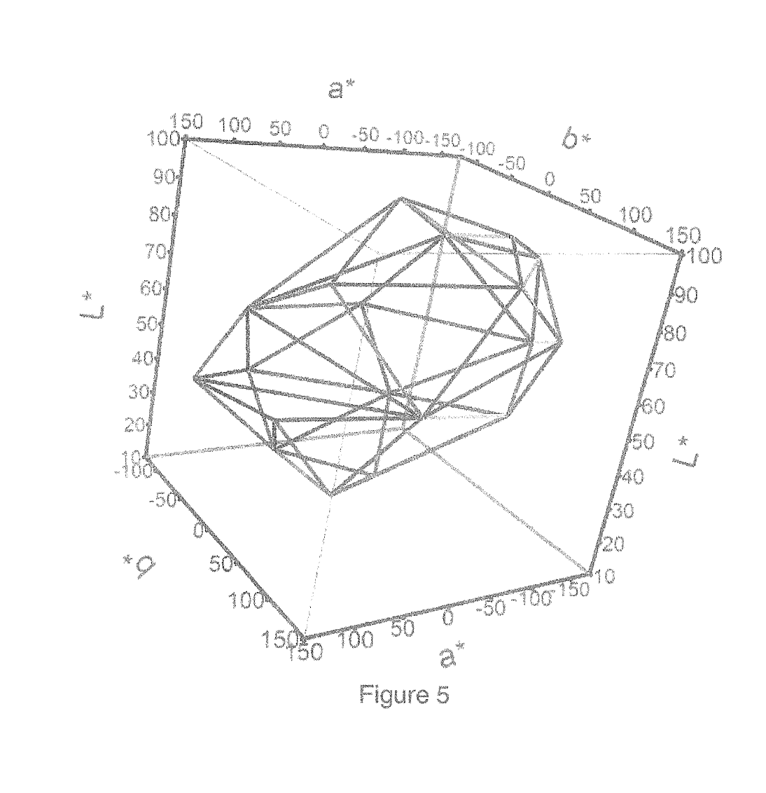

For the 3-D color gamuts discussed herein, the formula (new gamut volume—prior art gamut volume)/prior art gamut volume*100% is used to calculate the percent increase of the volume enveloped by the 3-D gamut plots of the MacAdam color gamut (FIGS. 4 and 5) (the maximum 3-D theoretical human color perception) compared to the Backsheet color gamut (FIGS. 2 and 3). The volume enveloped by the Backsheet 3-D color gamut and the MacAdam 3-D color gamut can be determined to be 339,777 and 2,572,500 relative volume units, respectively. Using these values in the equation results in 3-D color gamut percentage increases of about 657% (MacAdam), that is available over the palette of the prior art absorbent paper products—clearly, a surprising result.

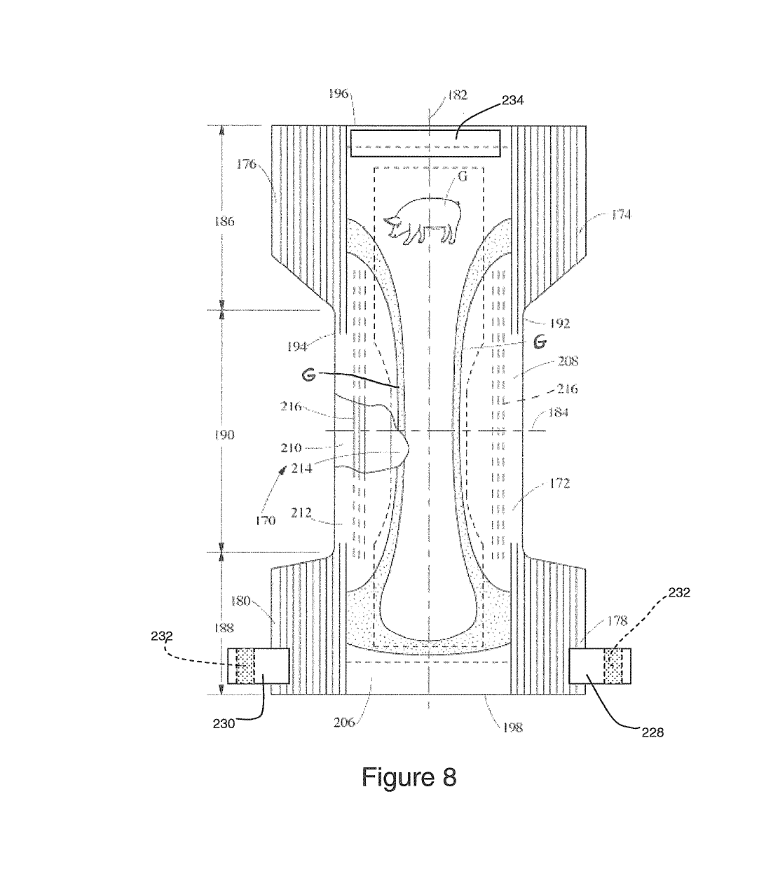

FIG. 1a shows an exemplary extrapolated graphical representation of the 2-dimensional (2-D) color gamut available to prior art fastening tabs substrates in an L*a*b color space in the a*b* plane. Exemplary embodiments of fastening tabs are described below with reference to FIG. 8. The L*a*b* points are chosen according to the Color Test Method described below. Without wishing to be limited by theory, it is thought that the most “intense” (i.e., 100% halftone) colors represent the outer boundaries of the color gamut. Surprisingly, it was found that the prior art Fastening Tabs (“FT”) 2-D color gamut 20 does not occupy as large of an area as the MacAdam 2-D color gamut 30 (the maximum 2-D theoretical human color perception) as applied to absorbent article fastening tab substrates of the present disclosure. Stated differently, the combination of the colors available with the MacAdam color gamut 30 provide resultant colors that extend beyond the limitations of the red, green, and blue-violet process colors and well beyond the Fastening Tabs 2-D color gamut 20 colors and color combinations when described in L*a*b* space.

For the 2-D color gamuts discussed herein, the formula (new gamut area—prior art gamut area)/prior art gamut area*100% is used to calculate the percent increase of the area circumscribed by the 2-D gamut plots of the MacAdam color gamut 30 compared to the Fastening Tabs color gamut 20. The area circumscribed by the Fastening Tabs color gamut 20 and the MacAdam color gamut 30 can be determined to be 1,449 and 45,100 relative area units, respectively. Using these values in the equation results in a color gamut percentage increase of about 3012% (MacAdam), that is available over the palette of the prior art absorbent paper products—clearly, a surprising result.

For the 3-D color gamuts discussed herein, the formula (new gamut volume—prior art gamut volume)/prior art gamut volume*100% is used to calculate the percent increase of the volume enveloped by the 3-D gamut plots of the MacAdam color gamut (FIGS. 4 and 5) (the maximum 3-D theoretical human color perception) compared to the Fastening Tabs color gamut (FIGS. 2a and 3a). The volume enveloped by the Fastening Tabs 3-D color gamut and the MacAdam 3-D color gamut can be determined to be 18,054 and 2,572,500 relative volume units, respectively. Using these values in the equation results in 3-D color gamut percentage increases of about 14,149% (MacAdam), that is available over the palette of the prior art absorbent paper products—clearly, a surprising result.

As described herein, in the context of absorbent paper products, it is observed that a product having the herein described increased color gamut are more visually perceptible when compared to products limited by the prior art gamut. This can be particularly true for absorbent paper products using the herein described gamuts. Without desiring to be bound by theory, this can be because there are more visually perceptible colors in the gamuts of the present disclosure. It is surprisingly noticed that the present invention also provides products having a full color scale with no loss in gamut.

The color gamut boundaries in both 2-D CIELab (L*a*b*) space and 3-D CIELab (L*a*b*) space commensurate in scope with the present disclosure may be approximated by the following system of equations in CIELab coordinates (L*a*b) respectively: MacAdam 2-D Color Gamut

{a*=−54.1 to 72.7;b*=131.5 to 145.8}→b*=0.113a*+137.6

{a*=−131.6 to −54.1;b*=89.1 to 131.5}→b*=0.547a*+161.1

{a*=−165.6 to −131.6;b*=28.0 to 89.1}→b*=1.797a*+325.6

{a*=3.6 to −165.6;b*=−82.6 to 28.0}→b*=−0.654a*−80.3

{a*=127.1 to 3.6;b*=−95.1 to −82.6}→b*=−0.101a*−82.3

{a*=72.7 to 127.1;b*=145.8 to −95.1}→b*=−4.428a*+467.7

wherein L* is from 0 to 100.

MacAdam 3-D Color Gamut (FIGS. 4 and 5)

| Vertexes defining each Face | | | | | | | | | | | | |
|---|---|---|---|---|---|---|---|---|---|---|---|---|
| Vertex 1 | | | Vertex 2 | | | Vertex 3 | | | E a* + F b* + G L* + H = 0 | | | |
| z1 | x1 | y1 | z2 | x2 | y2 | z3 | x3 | y3 | Face Plane Equation Coefficients | | | |
| L* | a* | b* | L* | a* | b* | L* | a* | b* | E | F | G | H |
| 20 | 41.6 | 24 | 20 | −24.6 | 4.3 | 20 | 48.9 | −58.2 | 0.0 | 0.0 | 5585.5 | −111709.0 |
| 20 | 41.6 | 24 | 20 | −24.6 | 4.3 | 37.8 | −162 | 25 | −350.7 | 1178.4 | −4077.1 | 67849.2 |
| 20 | 41.6 | 24 | 20 | 48.9 | −58.2 | 37.8 | 92.4 | −8.8 | −1463.2 | −129.9 | 3936.3 | −14740.4 |
| 20 | 41.6 | 24 | 37.8 | 92.4 | −8.8 | 61.7 | 72.7 | 146 | −3535.8 | −1564.8 | 7207.5 | 40493.6 |
| 20 | 41.6 | 24 | 37.8 | −162 | 25 | 61.7 | 72.7 | 146 | −2126.3 | 9043.7 | −24829.6 | 367998.5 |
| 20 | −24.6 | 4.3 | 20 | 48.9 | −58.2 | 37.8 | −63 | −38.1 | −1112.5 | −1308.3 | −5516.4 | 88586.2 |
| 20 | −24.6 | 4.3 | 37.8 | −63 | −38.1 | 37.8 | −162 | 25 | −1123.2 | −1762.2 | −6620.6 | 112360.0 |
| 20 | 48.9 | −58.2 | 37.8 | 92.4 | −8.8 | 37.8 | 127 | −95.1 | 1536.1 | 617.7 | −5468.2 | 70195.2 |
| 20 | 48.9 | −58.2 | 37.8 | 127 | −95.1 | 37.8 | 60.8 | −105 | 181.6 | −1180.1 | −3244.1 | −12680.2 |
| 20 | 48.9 | −58.2 | 37.8 | 60.8 | −105 | 37.8 | −63 | −38.1 | −1196.2 | −2203.6 | −5031.3 | 30866.4 |
| 37.8 | 92.4 | −8.8 | 37.8 | 127 | −95.1 | 61.7 | 72.7 | 146 | −2062.6 | −829.3 | 3664.5 | 44764.9 |
| 37.8 | 127 | −95.1 | 37.8 | 60.8 | −105 | 61.7 | 102 | −63 | −243.8 | 1584.6 | −2385.3 | 271840.3 |
| 37.8 | 127 | −95.1 | 61.7 | 72.7 | 146 | 61.7 | 102 | −63 | 4990.3 | 697.9 | 4324.4 | −731365.1 |
| 37.8 | 60.8 | −105 | 37.8 | −63 | −38.1 | 61.7 | −30.2 | −66 | 1606.1 | 2958.8 | 1249.9 | 166669.4 |
| 37.8 | 60.8 | −105 | 61.7 | 102 | −63 | 61.7 | −30.2 | −66 | 71.7 | −3157.2 | 5464.5 | −543370.7 |
| 37.8 | −63 | −38.1 | 37.8 | −162 | 25 | 61.7 | −161 | 33.4 | 1508.1 | 2366.1 | −888.4 | 218739.2 |
| 37.8 | −63 | −38.1 | 61.7 | −161 | 33.4 | 61.7 | −30.2 | −66 | 2375.7 | 3128.5 | 391.8 | 254053.1 |
| 37.8 | −162 | 25 | 61.7 | −161 | 33.4 | 69.5 | −132 | 89.1 | −1265.7 | 698.0 | −197.7 | −215023.8 |
| 37.8 | −162 | 25 | 69.5 | −132 | 89.1 | 61.7 | 72.7 | 146 | −2297.4 | 6713.4 | −11372.0 | −110150.0 |
| 61.7 | −161 | 33.4 | 69.5 | −132 | 89.1 | 91.7 | −83.2 | 85.3 | 1266.2 | −277.4 | −2808.0 | 386498.5 |
| 61.7 | −161 | 33.4 | 91.7 | −83.2 | 85.3 | 87 | −67.3 | −13.3 | 2714.1 | 843.1 | −8506.2 | 933905.6 |
| 61.7 | −161 | 33.4 | 87 | −67.3 | −13.3 | 61.7 | −30.2 | −66 | 2514.8 | 3311.8 | −3210.7 | 492624.0 |
| 69.5 | −132 | 89.1 | 91.7 | −83.2 | 85.3 | 91.7 | −1.2 | 145 | −1332.0 | 1820.4 | 3215.6 | −560973.0 |
| 69.5 | −132 | 89.1 | 91.7 | −1.2 | 145 | 61.7 | 72.7 | 146 | −1697.1 | 5552.6 | −4088.0 | −433958.6 |
| 91.7 | −83.2 | 85.3 | 91.7 | −1.2 | 145 | 98 | −33.9 | 95.7 | 378.0 | −516.6 | −2105.2 | 268562.4 |
| 91.7 | −83.2 | 85.3 | 98 | −33.9 | 95.7 | 87 | −67.3 | −13.3 | 572.3 | 331.9 | −5026.3 | 480221.4 |
| 91.7 | −1.2 | 145 | 98 | −33.9 | 95.7 | 98 | 8.3 | 3.3 | 582.1 | 265.9 | 5114.6 | −506939.7 |
| 91.7 | −1.2 | 145 | 61.7 | 72.7 | 146 | 76.1 | 67.7 | 4.6 | −4228.8 | −914.2 | −10432.2 | 1084383.8 |
| 91.7 | −1.2 | 145 | 76.1 | 67.7 | 4.6 | 98 | 8.3 | 3.3 | −3101.6 | −582.3 | −8447.2 | 855485.6 |
| 98 | −33.9 | 95.7 | 87 | −67.3 | −13.3 | 98 | 8.3 | 3.3 | −1016.4 | −464.2 | 7686.0 | −743256.1 |
| 87 | −67.3 | −13.3 | 61.7 | 102 | −63 | 98 | 8.3 | 3.3 | −126.7 | −3773.9 | 6566.0 | −629966.3 |
| 87 | −67.3 | −13.3 | 61.7 | 102 | −63 | 61.7 | −30.2 | −66 | −75.9 | 3342.1 | −7073.0 | 654690.6 |
| 61.7 | 72.7 | 146 | 61.7 | 102 | −63 | 76.1 | 67.7 | 4.6 | −3006.7 | −420.5 | −5167.0 | 598700.9 |
| 61.7 | 102 | −63 | 76.1 | 67.7 | 4.6 | 98 | 8.3 | 3.3 | 1499.2 | −106.4 | 4059.9 | −409962.2 |

The above-described 2-D color gamuts can be approximated by drawing straight lines to between the outermost points of the respective MacAdam color gamut 30 and Backsheet color gamut 10 as shown in FIG. 1. As shown, the 2-D Backsheet color gamut 10 absorbent paper products occupies a smaller CIELab (L*a*b*) color space than the 2-D MacAdam color gamut 30. In one non-limiting embodiment, the present disclosure provides for an absorbent article including a web substrate and/or component, such as a backsheet, topsheet, landing zone, fastener, ear, side panel, absorbent core, and/or acquisition layer, having a printed indicia comprising colors which may be described in the 2-dimensional a*b* axes of the CIELab (L*a*b*) color space extending between the area enclosed by the system of equations describing the MacAdam color gamut 30 and Backsheet color gamut 10 where L*=0 to 100.

In yet another exemplary, but non-limiting embodiment, the present disclosure provides for an absorbent article including a web substrate and/or component, such as a backsheet, topsheet, landing zone, fastener, ear, side panel, absorbent core, and/or acquisition layer, having a printed indicia comprising colors which may be described in the 3-dimensional CIELab (L*a*b*) color space extending between the area enclosed by the system of 3-D equations describing the MacAdam (FIGS. 4 and 5) and Backsheet color gamut (FIGS. 2 and 3) discussed herein.

The above-described 2-D color gamuts can be approximated by drawing straight lines to between the outermost points of the respective MacAdam color gamut 30 and Fastening Tabs color gamut 20 as shown in FIG. 1a. As shown, the 2-D Fastening Tabs color gamut 20 absorbent paper products occupies a smaller CIELab (L*a*b*) color space than the 2-D MacAdam color gamut 30. In one non-limiting embodiment, the present disclosure provides for an absorbent article including a web substrate and/or component, such as a fastening tab, having a body facing and/or garment facing surface completely printed and/or having a printed indicia with colors which may be described in the 2-dimensional a*b* axes of the CIELab (L*a*b*) color space extending between the area enclosed by the system of equations describing the MacAdam color gamut 30 and Fastening Tabs color gamut 20 where L*=0 to 100.

In yet another exemplary, but non-limiting embodiment, the present disclosure provides for an absorbent article including a web substrate and/or component, such as a fastening tab, having a body facing and/or garment facing surface completely printed and/or having a printed indicia comprising colors which may be described in the 3-dimensional CIELab (L*a*b*) color space extending between the area enclosed by the system of 3-D equations describing the MacAdam (FIGS. 4 and 5) and Fastening Tabs color gamut (FIGS. 2a and 3a) discussed herein.

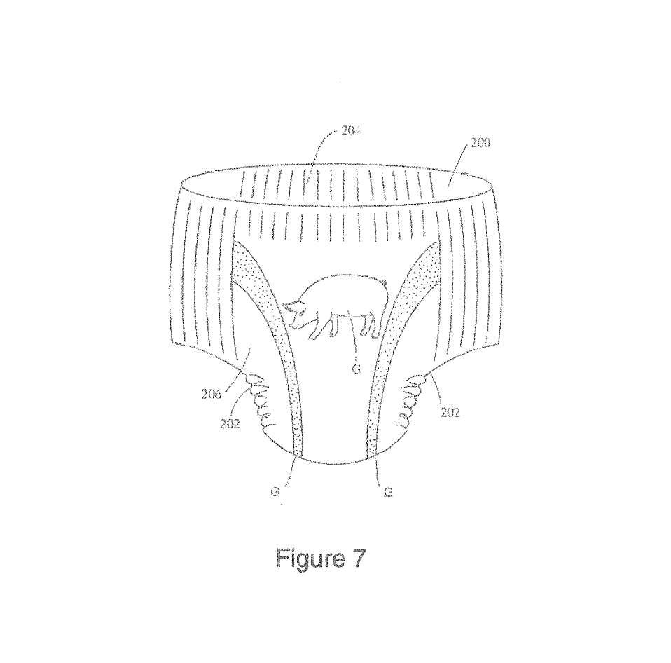

As mentioned above, the printing apparatuses and processes disclosed herein may be used to print indicia on web materials used to produce components of absorbent articles, such as diapers. For the purposes of a specific illustration, FIG. 7 shows one example of a disposable absorbent article in the form of a diaper 170 which may be substrates and/or components having printed indicia as described above. FIG. 8 is a plan view of the diaper 170 including a chassis 172 shown in a flat, unfolded condition, with the portion of the diaper that faces away from a wearer oriented towards the viewer. A portion of the chassis structure is cut-away in FIG. 8 to more clearly show the construction of and various features that may be included in embodiments of the diaper.

As shown in FIG. 8, the diaper 170 includes a 172 chassis having a first ear 174, a second ear 176, a third ear 178, and a fourth ear 180. To provide a frame of reference for the present discussion, the chassis 172 is shown with a longitudinal axis 182 and a lateral axis 184. The chassis 172 is shown as having a first waist region 186, a second waist region 188, and a crotch region 190 disposed intermediate the first and second waist regions. The periphery of the diaper is defined by a pair of longitudinally extending side edges 192, 194; a first outer edge 196 extending laterally adjacent the first waist region 186; and a second outer edge 198 extending laterally adjacent the second waist region 188. As shown in FIG. 7, the diaper 170 has a waist opening 200 and two leg openings 202.

As shown in FIGS. 7 and 8, the chassis includes an inner, body facing surface 204, and an outer, garment facing surface 206. As shown in FIG. 8, the chassis 172 may include an outer covering layer 208 including a topsheet 210 and a backsheet 212. An absorbent core 214 may be disposed between a portion of the topsheet 210 and the backsheet 212. It is to be appreciated that any one or more of the regions of the chassis may be stretchable and may include various types of elastomeric materials and/or laminates. As such, the diaper may be configured to adapt to a specific wearer's anatomy upon application and to maintain coordination with the wearer's anatomy during wear.

The diaper 170 may also be provided in the form of a pant-type diaper or may alternatively be provided with a re-closable fastening system, which may include fastener elements in various locations to help secure the diaper in position on the wearer. Fastener elements may be located on the ears and may be adapted to releasably connect with one or more corresponding fastening elements located in the first or second waist regions. For example, as shown in FIG. 8, the diaper 170 includes a first fastening tab 228 and a second fastening tab 230 connected with the chassis 172 in the second waist region 188. As shown in FIG. 8, the fastening tabs 228, 230 are connected along distal end regions of the third ear 178 and fourth ear 180, respectively. The fastening tabs 228, 230 may also include various types of releasably engageable fasteners and/or refastenable fastening structures. For example, the first and second fastening tabs 228, 230 may include mechanical fasteners, 232, in the form of hook and loop fasteners, hook and hook fasteners, macrofasteners, buttons, snaps, tab and slot fasteners, tape fasteners, adhesive fasteners, cohesive fasteners, magnetic fasteners, hermaphrodidic fasteners, and the like. Some examples of fastening systems and/or fastening tabs 228, 230 are discussed in U.S. Pat. Nos. 3,848,594; 4,662,875; 4,846,815; 4,894,060; 4,946,527; 5,151,092; 5,221,274; 6,251,097; 6,669,618; 6,432,098; and U.S. Patent Publication Nos. 2007/0078427 and 2007/0093769.

The fastening tabs 228, 230 may be adapted to releasably and/or refastenably engage or connect with another portion of the diaper 170. For example, as shown in FIG. 8, the diaper 170 may include a connection zone 234, sometimes referred to as a landing zone, in the first waist region 186. As such, when the taped diaper 170 is placed on a wearer, the fastening tabs 228, 230 are pulled around the waist of the wearer and connected with the connection zone 234 in the first waist region 186 to form a closed waist circumference and a pair of laterally opposing leg openings. It is to be appreciated that the connection zone may be constructed from a separate substrate that is connected with the chassis 172 of the diaper. In some embodiments, the connection zone may be integrally formed as part of the backsheet 212 of the diaper 170 or may be formed as part of the first and second ears in one or both of the waist regions, such as described in U.S. Pat. Nos. 5,735,840 and 5,928,212.

The diaper 170 may further include a non-engagement zone disposed on the same surface and in the same waist region as the fastening tabs 228, 230. The non-engagement zone may be configured to help prevent the fastening tabs 228, 230 from becoming engaged with other elements of the absorbent article. The non-engagement zone may comprise a film, coating or other material that does not attach to or engage with the fastening tabs 228, 230. In certain embodiments the non-engagement zone is in surface to surface contact with the fastening surface of the fastening component when the diaper 170 is packaged.

Embodiments of the diaper may also include pockets for receiving and containing waste, spacers which provide voids for waste, barriers for limiting the movement of waste in the article, compartments or voids which accept and contain waste materials deposited in the diaper, and the like, or any combinations thereof. Examples of pockets and spacers for use in absorbent products are described in U.S. Pat. Nos. 5,514,121; 5,171,236; 5,306,266; 5,397,318; 5,540,671; and PCT Patent Publication No. WO 93/25172, which are all hereby incorporated by reference herein. Examples of compartments or voids are disclosed in U.S. Pat. Nos. 4,968,312; 4,990,147; 5,062,840; 6,482,191; and 5,269,755, which are all hereby incorporated by reference herein. Examples of suitable transverse barriers are described in U.S. Pat. Nos. 5,554,142 and 5,653,703, and PCT Patent Publication No. WO 94/14395, which are all hereby incorporated by reference herein. All of the above-cited references are hereby incorporated by reference herein. In addition to or in place of the voids, pockets and barriers, described above, embodiments of the absorbent article may also include a waste management element capable of effectively and efficiently accepting, storing and/or immobilizing viscous fluid bodily waste, such as runny feces, such as described in U.S. Pat. No. 6,010,491, which is hereby incorporated by reference herein.

As previously mentioned, the chassis 172 may include the backsheet 212, shown for example, in FIG. 8. In some embodiments, the backsheet is configured to prevent exudates absorbed and contained within the chassis from soiling articles that may contact the diaper, such as bedsheets and undergarments. Some embodiments of the backsheet may be fluid permeable, while other embodiments may be impervious to liquids (e.g., urine) and comprises a film, such as a thin plastic film. In some embodiments, the plastic film includes a thermoplastic film having a thickness of about 0.012 mm (0.5 mil) to about 0.051 mm (2.0 mils). Some backsheet films may include those manufactured by Tredegar Industries Inc. of Terre Haute, Ind. and sold under the trade names X15306, X10962, and X10964. Other backsheet materials may include breathable materials that permit vapors to escape from the diaper while still preventing exudates from passing through the backsheet. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Publication No. WO 95/16746 and U.S. Pat. No. 5,865,823, both of which are hereby incorporated by reference herein. Other breathable backsheets including nonwoven webs and apertured formed films are described in U.S. Pat. Nos. 5,571,096 and 6,573,423; which are all hereby incorporated by reference herein.

The backsheet 212 may be formed by only one sheet (or layer) material such as a breathable (or microporous) film material or a non-breathable (or non-microporous) film material. In some embodiments, the backsheet may be formed by two (or more) sheet (or layer) materials which may include a non-breathable (or breathable) film material and a nonwoven outer cover material. In some embodiments, the backsheet may be formed by a laminate of two sheet (or layer) materials joined together, for example, the backsheet may include a non-breathable film material and a nonwoven material which is joined to the garment facing surface of the film material to provide a cloth-like and/or garment-like feel. In accordance with the discussion above, indicia may be printed on a substrate to make printed component material, which may be converted into printed components to manufacture the backsheet. Thus, the substrate may be in the form of a film material and/or nonwoven material used to construct the backsheet. As such, indicia G may be printed on any surface of the component material(s) of the backsheet. For example, indicia can be printed on any of the garment facing surfaces and the body facing surfaces of the film material and the nonwoven material. In some embodiments, indicia are printed directly on the nonwoven material. In other embodiments, the indicia G are printed on the garment facing surface of the film material. In such an arrangement, the indicia may be covered (or protected) by the nonwoven material, wherein the indicia are visible through the nonwoven material.

As with the backsheet 212, indicia may be printed on a substrate used as a printed component material to construct the topsheet 210. As such, indicia G may be printed on any surface of the component material(s) of the topsheet. The topsheet may be constructed to be compliant, soft feeling, and non-irritating to the wearer's skin. Further, all or at least a portion of the topsheet may be liquid pervious, permitting liquid to readily penetrate therethrough. As such, the topsheet may be manufactured from a wide range of materials, such as porous foams; reticulated foams; apertured nonwovens or plastic films; or woven or nonwoven webs of natural fibers (e.g., wood or cotton fibers), synthetic fibers (e.g., polyester, polyethylene, or polypropylene fibers), or a combination of natural and synthetic fibers. If the absorbent assemblies include fibers, the fibers may be spunbonded, carded, wet-laid, meltblown, hydroentangled, or otherwise processed as is known in the art. One example of a topsheet including a web of staple length polypropylene fibers is manufactured by Veratec, Inc., a Division of International Paper Company, of Walpole, Mass. under the designation P-8.

Examples of formed film topsheets are described in U.S. Pat. Nos. 3,929,135; 4,324,246; 4,342,314; 4,463,045; and 5,006,394, all of which are hereby incorporated by reference herein. Other topsheets may be made in accordance with U.S. Pat. Nos. 4,609,518 and 4,629,643, which are hereby incorporated by reference herein. Such formed films are available from The Procter & Gamble Company of Cincinnati, Ohio as “DRI-WEAVE” and from Tredegar Corporation of Terre Haute, Ind. as “CLIFF-T.”

In some embodiments, the topsheet is made of a hydrophobic material or is treated to be hydrophobic in order to isolate the wearer's skin from liquids contained in the absorbent core. If the topsheet is made of a hydrophobic material, at least the upper surface of the topsheet may be treated to be hydrophilic so that liquids will transfer through the topsheet more rapidly. This diminishes the likelihood that body exudates will flow off the topsheet rather than being drawn through the topsheet and being absorbed by the absorbent core. The topsheet can be rendered hydrophilic by treating it with a surfactant or by incorporating a surfactant into the topsheet. Suitable methods for treating the topsheet with a surfactant include spraying the topsheet material with the surfactant and immersing the material into the surfactant. A more detailed discussion of such a treatment and hydrophilicity is contained in U.S. Pat. Nos. 4,988,344 and 4,988,345, which are hereby incorporated by reference herein. A more detailed discussion of some methods for incorporating surfactant in the topsheet can be found in U.S. Statutory Invention Registration No. H1670, which is incorporated by reference herein.

In some embodiments, the topsheet may include an apertured web or film that is hydrophobic. This may be accomplished eliminating the hydrophilizing treatment step from the production process and/or applying a hydrophobic treatment to the topsheet, such as a polytetrafluoroethylene compound like SCOTCHGUARD or a hydrophobic lotion composition, as described below. In such embodiments, the apertures may be large enough to allow the penetration of aqueous fluids like urine without significant resistance. A more detailed discussion of various apertured topsheets can be found in U.S. Pat. Nos. 5,342,338; 5,941,864; 6,010,491; and 6,414,215, all of which are hereby incorporated by referenced herein.

Any portion of the topsheet may be coated with a lotion, such as topsheets described in U.S. Pat. Nos. 5,607,760; 5,609,587; 5,635,191; 5,643,588; and U.S. Pat. No. 6,498,284, all of which are hereby incorporated by reference herein. The lotion may function alone or in combination with another agent as the hydrophobizing treatment described above. The topsheet may also include or be treated with antibacterial agents, some examples of which are disclosed in PCT Publication No. WO 95/24173, which is hereby incorporated by reference herein. Further, the topsheet, the backsheet, or any portion of the topsheet or backsheet may be embossed and/or matte finished to provide a more cloth-like appearance.

The absorbent core 214 may include components such as an acquisition layer and absorbent material that is generally compressible, conformable, non-irritating to the wearer's skin, and capable of absorbing and retaining liquids such as urine and other body exudates. Thus, in addition to backsheet and topsheet components, it should be appreciated that indicia may be printed on substrates used as printed component material to construct the absorbent core and acquisition layer. In addition, indicia G may be printed on any surface of various component material(s) of the absorbent core. The absorbent core can also be manufactured in a wide variety of sizes and shapes (e.g., rectangular, hourglass, T-shaped, asymmetric, etc.). The absorbent core may also include a wide variety of liquid-absorbent materials commonly used in disposable diapers and other absorbent articles. In one example, the absorbent core includes comminuted wood pulp, which is generally referred to as airfelt. Examples of other absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

It is to be appreciated that the configuration and construction of the absorbent core may be varied (e.g., the absorbent core(s) or other absorbent structure(s) may have varying caliper zones, a hydrophilic gradient, a superabsorbent gradient, or lower average density and lower average basis weight acquisition zones; or may comprise one or more layers or structures). Exemplary absorbent structures are described in U.S. Pat. Nos. 4,610,678; 4,673,402; 4,834,735; 4,888,231; 5,137,537; 5,147,345; 5,342,338; 5,260,345; 5,387,207; and 5,650,222, all of which are hereby incorporated by reference herein.

The absorbent core may also have a multiple layered construction. A more detailed discussion of various types of multi-layered absorbent cores can be found in U.S. Pat. Nos. 5,669,894; 6,441,266; and 5,562,646; European Patent No. EP0565606B1; U.S. Patent Publication Nos. 2004/0162536A1 and 2004/0167486A1; and PCT Publication No. WO 2006/015141, which are all hereby incorporated by reference herein. In some embodiments, the absorbent article includes an absorbent core that is stretchable. In such a configuration, the absorbent core may be adapted to extend along with other materials of the chassis in longitudinal and/or lateral directions. The absorbent core can also be connected with the other components of the chassis various ways. For example, the diaper may include a “floating core” configuration or a “bucket” configuration wherein the diaper includes an anchoring system that can be configured to collect forces tending to move the article on the wearer. Such an anchoring system can also be configured to anchor itself to a body of a wearer by contacting various parts of the body. In this way, the anchoring system can balance the collected moving forces with holding forces obtained from the anchoring. By balancing the collected moving forces with the obtained holding forces, the anchoring system can at least assist in holding the disposable wearable absorbent article in place on a wearer. A more detailed discussion of various floating and/or bucket core configurations can be found in U.S. Patent Publication Nos. 2007/0287981 A1; 2007/0287982 A1; and 2007/0287983 A1, which are all hereby incorporated by reference herein.

The diapers according to the present disclosure can also include other features such as elastically extensible side panels. The side panels may be joined at seams to form the waist opening and the leg openings. The diapers may also includes leg elastics 216, such as shown in FIG. 8, and an elastic waist region to enhance the fits around the legs and waist of the wearer. Example leg elastic and leg cuff embodiments are disclosed in, for example, U.S. Pat. Nos. 4,695,278 and 4,795,454.

In addition to the backsheet, topsheet, absorbent core, acquisition layer, and other diaper components, indicia may also be printed on substrates used as printed component material to construct the fastening elements on the diaper, such as for example, a landing zone. As discussed above, depending on the particular configuration, it is to be appreciated that various types of fastening elements may be used with the diaper. In one example, the fastening elements include hook & loop fasteners, such as those available from 3M or Velcro Industries. In other examples, the fastening elements include adhesives and/or tap tabs, while others are configured as a macrofastener or hook (e.g., a MACRO or “button-like” fastener). Some exemplary fastening elements and systems are disclosed in U.S. Pat. Nos. 3,848,594; 4,662,875; 4,846,815; 4,894,060; 4,946,527; and 5,151,092, which are all hereby incorporated by reference herein. Additional examples of fasteners and/or fastening elements are discussed in U.S. Pat. Nos. 6,482,191; 6,251,097; and 6,432,098; U.S. Patent Publication Nos. 2007/0078427 A1 and 2007/0093769 A1, which are all hereby incorporated by reference herein. Other fastening systems are described in more detail in U.S. Pat. Nos. 5,595,567; 5,624,427; 5,735,840; and 5,928,212, which are all hereby incorporated by reference herein. The fastening system may also provide a means for holding the article in a disposal configuration as disclosed in U.S. Pat. No. 4,963,140, which is hereby incorporated by reference herein.

The foregoing description of the diaper shown in FIGS. 7 and 8, illustrate that indicia G may be printed according to the methods and apparatuses disclosed herein on substrates to construct various components, such as for example, backsheets, topsheets, absorbent cores, acquisition layers, landing zones, fastening tabs, and other fastening elements. In addition, the indicia may be printed on the body facing surface, the garment facing surface, or both surfaces of such components.

It is to be appreciated that the indicia disclosed herein may be in the form of graphics, such as images or designs that are constituted by a figure (e.g., a line(s)), a symbol or character, a color difference or transition of at least two colors, or the like. A graphic may include an aesthetic image or design that can provide certain benefit(s) when an absorbent article is viewed. It should also be appreciated that indicia may be in the form of permanent or active graphics. Active graphics are graphics that are configured to appear or disappear upon various types of triggering mechanisms or stimuli, such as for example, moisture (e.g. aquachromic ink graphics), temperature change (e.g. thermochromic ink graphics), and/or light (e.g. photochromic ink graphics, UV or IR light).

It is also to be appreciated that embodiments of absorbent articles disclosed herein and including printed indicia may have various average wet crockfastness values. For example, some embodiments of absorbent articles may have an average wet crockfastness value of at least 4 or greater. A method for measuring wet crockfastness values is disclosed in U.S. Pat. No. 5,458,590, which is hereby incorporated by reference.

### Analytical and Testing Procedures

### Color Test Method

CIELab (L*a*b*) values of a finally printed product produced according to the present disclosure discussed herein can be measured with a colorimeter, spectrophotometer, or spectrodensitometer according to ISO 13655. A suitable spectrodensitometer for use with this invention is the X-Rite 530 commercially available from X-Rite, Inc. of Grand Rapids, Mich.

Select the D50 illuminant and 2 degree observer as described. Use 45/0° measurement geometry. The spectrodensitometer should have a 10 nm measurement interval. The spectrodensitometer should have a measurement aperture of less than 2 mm. Before taking color measurements, calibrate the spectrodensitometer according to manufacturer instructions. Visible surfaces are tested in a dry state and at an ambient relative humidity of approximately 50%±2% and a temperature of 23° C.±1° C. Place the sample to be measured on a white backing that meets ISO 13655 section A3 specifications. Exemplary white backings are described on the web site: http://www.fogra.de/en/fogra-standardization/fogra-characterizationdata/information-about-measurement-backings/. Select a sample location on the visible surface of the printed product containing the color to be analyzed. The L*, a*, and b* values are read and recorded.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for making absorbent articles having a wide color gamut indicia printed thereon, the method comprising the steps of:
providing a topsheet;
providing a backsheet; and
disposing an absorbent core between the topsheet and the backsheet; and
printing an indicia directly on at least one of the backsheet, the absorbent core, and the topsheet, and wherein the indicia comprises halftone dots comprising five or more process colors comprising L*a*b* color values, the color values being defined by the difference in CIELab coordinate values disposed inside a first boundary described by the following system of equations:
{a*=−54.1 to 72.7;b*=131.5 to 145.8}→b*=0.113a*+137.6
{a*=−131.6 to −54.1;b*=89.1 to 131.5}→b*=0.547a*+161.1
{a*=−165.6 to −131.6;b*=28.0 to 89.1}→b*=1.797a*+325.6
{a*=3.6 to −165.6;b*=−82.6 to 28.0}→b*=−0.654a*−80.3
{a*=127.1 to 3.6;b*=−95.1 to −82.6}→b*=−0.101a*−82.3
{a*=72.7 to 127.1;b*=145.8 to −95.1}→b*=−4.428a*+467.7
wherein L* is from 0 to 100; and, the CIELab coordinate values disposed outside a second boundary described by the following system of equations:
{a*=−19.12 to −41.53;b*=−52.66 to −31.98}→b*=−0.9228a*−70.30
{a*=−41.53 to −46.41;b*=−31.98 to −25.51}→b*=−1.3258a*−87.04
{a*=−46.41 to −57.57;b*=−25.51 to 7.07}→b*=−2.9194a*−161.00
{a*=−57.57 to −60.55;b*=7.07 to 27.68}→b*=−6.9161a*−391.09
{a*=−60.55 to −59.35;b*=27.68 to 38.68}→b*=9.1667a*+582.72
{a*=−59.35 to −54.97;b*=38.68 to 55.49}→b*=3.8379a*+266.46
{a*=−54.97 to −6.32;b*=55.49 to 87.62}→b*=0.6604a*+91.79
{a*=−6.32 to 44.02;b*=87.62 to 64.58}→b*=−0.4577a*+84.73
{a*=44.02 to 59.83;b*=64.58 to 57.15}→b*=−0.4700a*+85.27
{a*=59.83 to 64.21;b*=57.15 to 52.64}→b*=−1.0297a*+118.76
{a*=64.21 to 65.33;b*=52.64 to 46.49}→b*=−5.4911a*+405.22
{a*=65.33 to 63.85;b*=46.49 to 30.88}→b*=10.5473a*−642.56
{a*=63.85 to 56.98;b*=30.88 to 1.59}→b*=4.2635a*−241.34
{a*=56.98 to 11.84;b*=1.59 to −39.9}→b*=0.9191a*−50.78
{a*=11.84 to −19.12;b*=−39.9 to −52.66}→b*=0.4121a*−44.78
wherein L* ranges from 0 to 100.

2. The method of claim 1, wherein the indicia is provided by process colors of cyan, yellow, magenta, black, red, green, and blue-violet.

3. The method of claim 1, further comprising the step of applying an anti-rub off coating.

4. The method of claim 1, further comprising the step of connecting a fastening tab with at least one of the topsheet and backsheet.

5. The method of claim 1, wherein the step of printing further comprises printing the indicia with a contact printing system adapted to print the 5 colors upon a web substrate utilizing 4 printing components.

6. A method for making absorbent articles having a wide color gamut indicia printed thereon, the method comprising the steps of:
providing a chassis;
connecting a fastening component with the chassis;
printing an indicia directly on one of the chassis and the fastening component, wherein the indicia comprises L*a*b* color values, the color values being defined by the difference in CIELab coordinate values disposed inside a first boundary described by the following system of equations:
{a*=−54.1 to 72.7;b*=131.5 to 145.8}→b*=0.113a*+137.6
{a*=−131.6 to −54.1;b*=89.1 to 131.5}→b*=0.547a*+161.1
{a*=−165.6 to −131.6;b*=28.0 to 89.1}→b*=1.797a*+325.6
{a*=3.6 to −165.6;b*=−82.6 to 28.0}→b*=−0.654a*−80.3
{a*=127.1 to 3.6;b*=−95.1 to −82.6}→b*=−0.101a*−82.3
{a*=72.7 to 127.1;b*=145.8 to −95.1}→b*=−4.428a*+467.7
wherein L* is from 0 to 100; and, the CIELab coordinate values disposed outside a second boundary described by the following system of equations:
{a*=−19.12 to −41.53;b*=−52.66 to −31.98}→b*=−0.9228a*−70.30
{a*=−41.53 to −46.41;b*=−31.98 to −25.51}→b*=−1.3258a*−87.04
{a*=−46.41 to −57.57;b*=−25.51 to 7.07}→b*=−2.9194a*−161.00
{a*=−57.57 to −60.55;b*=7.07 to 27.68}→b*=−6.9161a*−391.09
{a*=−60.55 to −59.35;b*=27.68 to 38.68}→b*=9.1667a*+582.72
{a*=−59.35 to −54.97;b*=38.68 to 55.49}→b*=3.8379a*+266.46
{a*=−54.97 to −6.32;b*=55.49 to 87.62}→b*=0.6604a*+91.79
{a*=−6.32 to 44.02;b*=87.62 to 64.58}→b*=−0.4577a*+84.73
{a*=44.02 to 59.83;b*=64.58 to 57.15}→b*=−0.4700a*+85.27
{a*=59.83 to 64.21;b*=57.15 to 52.64}→b*=−1.0297a*+118.76
{a*=64.21 to 65.33;b*=52.64 to 46.49}→b*=−5.4911a*+405.22
{a*=65.33 to 63.85;b*=46.49 to 30.88}→b*=10.5473a*−642.56
{a*=63.85 to 56.98;b*=30.88 to 1.59}→b*=4.2635a*−241.34
{a*=56.98 to 11.84;b*=1.59 to −39.9}→b*=0.9191a*−50.78
{a*=11.84 to −19.12;b*=−39.9 to −52.66}→b*=0.4121a*−44.78
wherein L* ranges from 0 to 100.

7. The method of claim 6, wherein the indicia comprises halftone dots.

8. The method of claim 7, wherein the halftone dots comprise five or more process colors.

9. The method of claim 8, wherein the indicia is provided by process colors of cyan, yellow, magenta, black, red, green, and blue-violet.

10. The method of claim 8, wherein the step of printing further comprises printing the indicia with a contact printing system adapted to print the 5 colors upon a web substrate utilizing 4 printing components.

11. The method of claim 6, wherein the chassis comprises a topsheet, a backsheet, and an absorbent core disposed between the topsheet and the backsheet.

12. The method of claim 11, further comprising the step of connecting a fastening tab with at least one of the topsheet and backsheet.

13. The method of claim 6, further comprising the step of applying an anti-rub off coating.
